(19) United States
(12) Patent Application Publication
(10) Pub. No.: US 2016/0040239 A1
(43) Pub. Date: Feb. 11, 2016

(54) METHODS FOR PREDICTING CARDIOVASCULAR RISKS AND RESPONSIVENESS TO STATIN THERAPY USING SNPS

(71) Applicant: Ohio State Innovation Foundation, Columbus, OH (US)

(72) Inventors: Wolfgang Sadee, Upper Arlington, OH (US); Audrey C. Papp, Columbus, OH (US)

(21) Appl. No.: 14/819,766

(22) Filed: Aug. 6, 2015

Related U.S. Application Data
(63) Continuation of application No. 13/985,728, filed on Sep. 24, 2013, now abandoned.
(60) Provisional application No. 61/525,818, filed on Aug. 21, 2011.
(60) Provisional application No. 61/443,233, filed on Feb. 15, 2011.

(51) Int. Cl. C12Q 1/68

(52) U.S. Cl. CPC C12Q 1/6883; C12Q 2600/156; C12Q 2600/112

(57) ABSTRACT

Methods and compositions for the effect of Cholesteryl ester transfer protein (CETP) polymorphisms on mRNA splicing, statin treatment outcome, response to CETP inhibitor drugs, and myocardial infarction risk are described.

### CROSS-REFERENCE TO RELATED APPLICATIONS

This application claims the priority to U.S. provisional applications Ser. No. 61/443,233 filed Feb. 15, 2011, and Ser. No. 61/525,818 filed Aug. 21, 2011, the entire disclosures of which are expressly incorporated herein by reference.

### STATEMENT REGARDING FEDERALLY SPONSORED RESEARCH

This invention was made with government support under NIH grants awarded by General Medical Sciences GM61390 and U01GM092655. The government has certain rights in the invention.

### SEQUENCE LISTING

The instant application contains a Sequence Listing which has been submitted via EFS-web and is hereby incorporated by reference in its entirety. The ASCII copy, created on Feb. 15, 2012, is named 604_{—}52590_SEQ_LIST_OSURF-11059.txt, and is 11,626 bytes in size.

### TECHNICAL FIELD AND INDUSTRIAL APPLICABILITY OF THE INVENTION

Described herein are methods for detecting Cholesteryl Ester Transfer Protein CETP polymorphisms that affect mRNA expression and splicing, statin treatment outcome, and myocardial infarction risk, and response to CETP inhibitors, a new class of compounds in development. Also described are markers useful for the detection of CETP polymorphisms that affect mRNA expression and splicing, statin treatment outcome and myocardial infarction risk. In certain embodiments, such markers are useful to determine treatment outcomes with CETP inhibitors. In particular, methods for determining which subjects will most benefit from treatment therapies that are affected by the marker/s are described.

### BACKGROUND

The Cholesteryl Ester Transfer Protein (CETP), also called plasma lipid transfer protein, is a plasma protein that facilitates the transport of cholesteryl esters and triglycerides between the lipoproteins. CETP shuttles cholesterol esters from high-density lipoprotein particles (HDL) to low density lipoproteins (LDL).

High CETP activity lowers the HDL/total cholesterol ratio, potentially increasing risk for coronary artery disease (CAD). Therefore, inhibition of CETP offers a new approach to CAD therapy. However, one CETP inhibitor, torcetrapib, was found to actually increase cardiovascular events, even though HDL increased and LDL decreased substantially. Other recent results further question the validity of the CETP-HDL-CAD relationship under all conditions, showing that low CETP levels can associate with increased CAD risk, possibly because of functions other than cholesterol transport.

The CETP gene is located on the long (q) arm of chromosome 16 at position 21; more precisely, the CETP gene is located from base pair 56,995,834 to base pair 57,017,755 on chromosome 16. CETP is highly polymorphic. CETP polymorphisms appear to affect cardiovascular risk and therapy in a sex-dependent manner, reflecting different lipid metabolism in males and females. Alternative splicing also affects CETP activity. An in-frame deletion of exon 9 ('9) generates a shorter '9 protein, which dimerizes with the full-length form preventing its efflux from the liver, possibly acting in a dominant negative manner. While production of the '9 splice variant is influenced by diet, genetic factors have yet to be determined.

In the field of human genomics, pharmacogenomics is generally considered to include efforts to use human DNA sequence variability in the development and prescription of drugs. Pharmacogenomics is based on the correlation or association between a given genotype and a resulting phenotype. Pharmacogenomics information can be especially useful in clinical settings where correlation information can be used to prevent drug toxicities, or develop treatment regimes. However, only a small percentage of observed drug toxicities have been explained adequately by the set of pharmacogcnomic markers available to date.

In addition, “outlier” subjects, or subjects experiencing unanticipated effects in clinical trials (when administered drugs that have previously been demonstrated to be both safe and efficacious), can cause substantial delays in obtaining FDA drug approval and may even cause certain drugs to come off market, though such drugs may be efficacious for a majority of recipients. Despite the current understanding that all humans are 99.9% identical in their genetic makeup (and the DNA sequence of any two subjects being nearly identical) there are variations between subjects. These include, for example, deletions or insertions of DNA sequences, variations in the number of repetitive DNA elements in non-coding regions and changes in a single nucleotide position, or “single nucleotide polymorphisms” (SNP). SNPs are useful as genetic markers for phenotypic traits. Phenotypic traits of particular interest to the medical field include predisposition to disease, and response to a particular drug or treatment regime.

It would be desirable and advantageous to have one or a combination of biomarkers which could be used to determine a subject's risk of disease, response to an agent, or the suitability of a subject to treatment with one or more agents using various doses and dose regimens.

### SUMMARY

The inventions described and claimed herein have many attributes and embodiments including, but not limited to, those set forth or described or referenced in this Summary. It is not intended to be all-inclusive and the inventions described and claimed herein are not limited to or by the features or embodiments identified in this Summary, which is included for purposes of illustration only and not restriction.

In a first broad aspect, there is provided herein a method for determining whether a human subject has an increased or reduced risk for developing a coronary artery disease (CAD), comprising:

testing nucleic acid from the human subject to determine the presence of at least one single nucleotide polymorphism (SNP) in the cholesteryl ester transfer protein (CLIP) gene,

the at least one SNP comprising one or more of:
a) rs247616 [SEQ ID NO:31], rs5883 [SEQ ID NO:34], rs3764261 [SEQ ID NO:33], and rs9930761 [SEQ ID NO:35],
b) one or more SNPs in linkage disequilibrium with the SNP/s of (a), and
c) combinations of (a) and (b);

wherein the presence of a minor allele of the SNP being detected indicates that the human subject has an altered risk for developing CAD.

In certain embodiments, the SNP is rs247616 [SEQ ID NO:31].

In certain embodiments, the SNP is rs5883 [SEQ ID NO:34].

In certain embodiments, the SNP is rs3764261 [SEQ ID NO:33].

In certain embodiments, the SNP is rs9930761 [SEQ ID NO:35].

In certain embodiments, the SNPs are rs247616 [SEQ ID NO:31] and rs5883 [SEQ ID NO:34].

In certain embodiments, the SNP are rs247616 [SEQ ID NO:31] and rs3764261 [SEQ ID NO:33].

In certain embodiments, the SNP are rs247616 [SEQ ID NO:31] and rs9930761 [SEQ ID NO:35].

In certain embodiments, the SNPs are rs5883 [SEQ ID NO:34] and rs9930761 [SEQ ID NO:35].

In certain embodiments, the SNPs are rs247616 [SEQ ID NO:31], rs5883 [SEQ ID NO:34], rs3764261 [SEQ ID NO:33], and rs9930761 [SEQ ID NO:35].

In certain embodiments, the testing step further comprises detecting whether the human subject is homozygous for the SNP.

In another broad aspect, there is provided herein a method of determining whether a human subject has a risk for developing a coronary artery disease (CAD), comprising: testing nucleic acid from the human subject for the presence or absence of:

T at a polymorphism in gene CETP at position +121 in Exon 9 (rs5883C>T [SEQ ID NO:34]), or A at such position in its complement,

wherein the presence of the T allele indicates that the human subject has an increased risk for CAD. In certain embodiments, the subject is already in a risk category (e.g., having hypertension).

In another broad aspect, there is provided herein a method of determining whether a human subject has a risk for developing a coronary artery disease (CAD), comprising: testing nucleic acid from the human subject for the presence or absence of:

T at a polymorphism in gene CETP at position −6152 (rs247616C>T[SEQ ID NO:31]), or A at such position in its complement;

wherein a homozygous presence of the T allele indicates that the human subject has a decreased risk for CAD. In certain embodiments, the subject is at devoid of other obvious risk factors.

In another broad aspect, there is provided herein a method of determining whether a human subject has a risk for developing a coronary artery disease (CAD), comprising: testing nucleic acid from the human subject for at least a heterozygous presence of:

T at a polymorphism in gene CETP at position −6152 (rs247616 [SEQ ID NO:31]), or C at such position in its complement; and,

C at a polymorphism in gene CETP at position −40 in Exon 8 (rs9930761T>C [SEQ ID NO:35]), or T at such position in its complement,

wherein such at least heterozygous presence at SNPs rs247616 [SEQ ID NO:31] and rs993071 indicates that the human subject has a decreased risk for CAD.

In another broad aspect, there is provided herein a method of determining whether a human subject has a risk for developing coronary artery disease (CAD), comprising: testing nucleic acid from the human subject for the presence or absence of:
C at a polymorphism in gene CETP at position −40 in Exon 8 (rs9930761 [SEQ ID NO:35]), or T at such position in its complement,
wherein the presence of the C indicates that the human subject has an increased risk for CAD in an at-risk population.

In certain embodiments, the CAD comprises at least: an altered response to a cholesterol ester transfer protein (CETP) inhibitor.

In certain embodiments, the CAD comprises one or more of: arterial disease, atheroma, atherosclerosis, arteriosclerosis, coronary artery disease, arrhythmia, angina pectoris, congestive heart disease, myocardial infarction, stroke, transient ischemic attack (TIA), aortic aneurysm, cardiopericarditis, infection and/or inflammation of heart tissue, vascular and clotting problems, insufficiencies, and combinations thereof.

In certain embodiments, at least one additional SNP of the CETP gene that is in high linkage disequilibrium with rs247616 [SEQ ID NO:31] is tested, wherein the at least one additional SNP is selected from several SNPs including: rs173539 [SEQ ID NO:32], rs3726432 [SEQ ID NO:53], and rs3764261[SEQ ID NO:33].

In certain embodiments, the at least one additional SNP of the CETP gene that is in high linkage disequilibrium with rs9930761 [SEQ ID NO:35] and/or rs5883 [SEQ ID NO:34] is tested, wherein the at least one additional SNP is selected from: rs12720873 [SEQ ID NO:36] and rs11644475 [SEQ ID NO:37].

In certain embodiments, the human subject is a male.

In certain embodiments, the human subject is a healthy human subject lacking other CAD risk factors.

In certain embodiments, the human subject is a human subject already progressing toward CAD with at least one other CAD risk factor.

In certain embodiments, the he human subject is evaluated for the human's medical history, family history, age, gender, socio-economic status, race, ethnicity, smoking, plasma cholesterol levels, plasma triglyceride levels, plasma lipids, plasma glucose, plasma insulin, plasma lipoproteins, or coronary artery calcification using electron-beam computer tomography.

In certain embodiments, the method comprises performing a procedure selected from: chain terminating sequencing, restriction digestion, allele-specific polymerase reaction, single-stranded conformational polymorphism analysis, genetic bit analysis, temperature gradient gel electrophoresis, ligase chain reaction, ligase/polymerase genetic bit analysis, allele specific hybridization, size analysis, nucleotide sequencing, 5′ nuclease digestion, primer specific extension, oligonucleotide microarray analysis, oligonucleotide ligation assay, or mass spectrophotometry.

In certain embodiments, the method comprises using a probe or primer that hybridizes under high stringency conditions to a nucleic acid sequence spanning the nucleotide.

In another broad aspect, there is provided herein a method for detecting risk for a coronary artery disease (CAD) in a human subject, comprising:

detecting in a nucleic acid sample obtained from the human or a genotype derived from the human the presence of an allele associated with increased risk for CAD wherein the allele is the presence of a “T” at the position of polymorphism identified by rs5883 [SEQ ID NO:34] in the cholesteryl ester transfer protein (CETP) gene; and

treating the human thus characterized for increased risk for CAD with therapy to delay onset of or slow progression of the CAD.

In another broad aspect, there is provided herein a method for detecting risk for a coronary artery disease (CAD) in a human subject, comprising:

detecting in a nucleic acid sample obtained from the human or a genotype derived from the human the presence of an allele associated with increased risk for CAD wherein the allele is the homozygous presence of a “T” at the position of polymorphism identified by rs247616 [SEQ ID NO:31] in the cholesteryl ester transfer protein (CETP) gene; and

treating the human thus characterized for increased risk for CAD with therapy to delay onset of or slow progression of the CAD.

In another broad aspect, there is provided herein a method for detecting risk for a coronary artery disease (CAD) in a human subject, comprising: detecting in a nucleic acid sample obtained from the human or a genotype derived from the human the presence of an allele associated with increased risk for CAD wherein the allele is the presence of a “C” at the position of polymorphism identified by rs9930761 [SEQ ID NO:35] in the cholesteryl ester transfer protein (CETP) gene; and,

treating the human thus characterized for increased risk for CAD with therapy to delay onset of or slow progression of the CAD

In certain embodiments, the treatment includes administering a drug, compound, biologic or combination thereof, that comprises a lipid-lowering agent.

In certain embodiments, the treatment includes administering a HMG-CoA reductase inhibitor.

In certain embodiments, the treatment includes administering a statin.

In certain embodiments, the treatment includes administering one or more of: atorvastatin, such as Lipitor®, Torvast®; atorvastatin+amlodipidine such as Besylate®; cerivastatin such as Lipobay®; cholystyramine; colestipol; fluvastatin such as Lescol®, Lescol XL®; gemfibrozil; lovastatin such as Mevacor®, Altocor®, Altoprev®; Lovastatin+niacin such as extended-release Advicor®; mevastatin such as Compactin®; Pitavastatin such as Livalo®, Pitava®; pravastatin such as Pravachol®, Selektine®, Lipostat®; , probucol; rosuvastatin such as Crestor®; simvastatin such as Zocor®, Lipex®; simvastatin+ezetimibe such as Vytorin®; and, simvastatin+niacin such as extended-release Simcor®.

In another broad aspect, there is provided herein a method comprising:

a) analyzing a sample from a human subject with a SNP detection assay to determine that the subject is at least heterozygous for one or more of polymorphisms SNP rs5883 [SEQ ID NO:34], rs247616 [SEQ ID NO:31] and rs9930761 [SEQ ID NO:35] in the CETP gene, thereby generating a genetic analysis result;

b) inputting the genetic analysis result into a system, wherein the system comprises:

i) a computer processor for receiving, processing, and communicating data,

ii) a storage component for storing data which contains a reference genetic database of results of at least one genetic analysis of the one or more SNPs in the CETP gene with respect to risk for coronary artery disease (CAD), and

iii) a computer program, embedded within the computer processor, which is configured to process the homozygous genetic analysis result in the context of the reference database to determine, as an outcome, whether the human subject has a risk for developing CAD;

c) processing the genetic analysis result with the computer program in the context of the reference database to determine, as an outcome, whether the human subject has a risk developing CAD;

d) communicating the outcome from the computer program; and optionally

e) administering to the human subject treatment for the prevention or amelioration of a coronary artery disease (CAD).

In certain embodiments, the method further comprises the step of analyzing the sample for the homozygous presence SNP rs247616 [SEQ ID NO:31].

In another broad aspect, there is provided herein a diagnostic kit comprising at least one reagent for detecting a risk marker for coronary artery disease (CAD), comprising: an array comprising a substrate carrying one or more reagents for identifying in a nucleic acid sample from a subject the occurrence of at least one SNP in the CETP gene that is associated with the risk of developing or expediting a CAD, wherein the at least one SNP comprises one or more of: rs247616 [SEQ ID NO:31], rs5883 [SEQ ID NO:34], rs3764261 [SEQ ID NO:33], and rs9930761 [SEQ ID NO:35].

In certain embodiments, the kit comprises at least one of the following components one or more primers for amplifying at one or more SNPs of the CETP gene; at least one enzyme for nucleic acid amplification; at least one enzyme suitable for performing an analysis for identifying at least one risk marker; and instructions for use.

In certain embodiments, the reagent of the array detects the single nucleotide polymorphism (SNP) rs5883 [SEQ ID NO:34] of the CETP gene.

In certain embodiments, the reagent of the array detects the single nucleotide polymorphism (SNP) rs247616 [SEQ ID NO:31] of the CETP gene.

In certain embodiments, the reagent of the array detects the single nucleotide polymorphism (SNP) rs9930761 [SEQ ID NO:35] of the CETP gene.

In certain embodiments, the reagent of the array detects the single nucleotide polymorphism (SNP) rs3764261 [SEQ ID NO:33] of the CETP gene

In another broad aspect, there is provided herein a diagnostic kit for detecting one or more coronary artery disease (CAD)-associated polymorphisms in a genetic sample, comprising at least one probe for assessing the presence of a single nucleotide polymorphism (SNP) selected from: rs247616 [SEQ ID NO:31], rs5883 [SEQ ID NO:34], rs3764261 [SEQ ID NO:33], and rs9930761 [SEQ ID NO:35].

In certain embodiments, the at least one probe is selected from the group of sense, anti-sense, and naturally occurring mutants, of any one of: rs247616 [SEQ ID NO:31], rs5883 [SEQ ID NO:34], rs3764261 [SEQ ID NO:33], and rs9930761 [SEQ ID NO:35].

In certain embodiments, at least one probe being from 3 to 101 nucleotides in length.

In certain embodiments, at least one probe being a length selected from the group of from about 5 to 101, from about 7 to 101, from about 9 to 101, from about 15 to 101, from about 20 to 101, from about 25 to 101, from about 30 to 101, from about 40 to 101, from about 50 to 101, from about 60 to 101, from about 70 to 101, from about 80 to 101, from about 90 to 101, and from about 99 to 101 nucleotides in length.

In another broad aspect, there is provided herein a microarray for detecting one or more coronary artery disease (CAD)-associated polymorphisms in a genetic sample, comprising at least one probe for assessing the presence of a single nucleotide polymorphism (SNP) in the cholesteryl ester transfer protein (CETP) gene selected from: rs247616 [SEQ ID NO:31], rs5883 [SEQ ID NO:34], rs3764261 [SEQ ID NO:33], and rs9930761 [SEQ ID NO:35].

In another broad aspect, there is provided herein a method of assessing a susceptibility to coronary artery disease (CAD) in a human subject, the method comprising a step of:

screening a sample from a human subject that comprises nucleic acid of the human subject for at least one polymorphism in CETP gene, that correlates with increased occurrence of myocardial infarction in a human population,

wherein the at least one polymorphism is selected from one or more of: rs247616 [SEQ ID NO:31], rs5883 [SEQ ID NO:34], rs3764261 [SEQ ID NO:33], and rs9930761 [SEQ ID NO:35]; wherein the presence of the at least one polymorphism in the nucleic acid identifies the subject as having elevated susceptibility to CAD, and

wherein the absence of the at least one polymorphism in the nucleic acid identifies the subject as not having the elevated susceptibility.

In another broad aspect, there is provided herein a method according to preceding claim, further comprising determining if the subject is male,

wherein the presence of the at least one polymorphism and a being male identifies the subject as having elevated susceptibility to CAD.

Benefit of Statin Rx

In another broad aspect, there is provided herein a method of identifying a human subject as having a deceased benefit from treatment with a statin for amelioration, or prevention, of coronary artery disease (CAD), comprising:

detecting, in a nucleic acid sample of the human subject, a T allele at single nucleotide polymorphism rs3764621 in the cholesteryl ester transfer protein (CETP) gene,

wherein the detection of a homozygous presence of the T allele of rs3764621 identifies the human subject as having a decreased benefit from statin treatment for coronary artery disease, and

wherein the decreased benefit is relative to a human subject that is not homozygous for the T allele at single nucleotide polymorphism rs3764261 [SEQ ID NO:33].

In certain embodiments, the human subject is a male.

In certain embodiments, the human subject is a healthy human subject lacking other CAD risk factors.

In certain embodiments, the human subject is a human subject already progressing toward CAD with at least one other CAD risk factor.

In certain embodiments, the human subject is evaluated for the human's medical history, family history, age, gender, socio-economic status, race, ethnicity, smoking, plasma cholesterol levels, plasma triglyceride levels, plasma lipids, plasma glucose, plasma insulin, plasma lipoproteins, or coronary artery calcification using electron-beam computer tomography.

In certain embodiments, the method comprises performing a procedure selected from: chain terminating sequencing, restriction digestion, allele-specific polymerase reaction, single-stranded conformational polymorphism analysis, genetic bit analysis, temperature gradient gel electrophoresis, ligase chain reaction, ligase/polymerase genetic bit analysis, allele specific hybridization, size analysis, nucleotide sequencing, 5′ nuclease digestion, primer specific extension, oligonucleotide microarray analysis, oligonucleotide ligation assay, or mass spectrophotometry

In certain embodiments, the method comprises using a probe or primer that hybridizes under high stringency conditions to a nucleic acid sequence spanning the nucleotide

In another broad aspect, there is provided herein a diagnostic kit comprising at least one reagent for detecting a risk marker for coronary artery disease (CAD), comprising: an array comprising a substrate carrying one or more reagents for identifying in a nucleic acid sample from a subject the occurrence of at least one SNP in the CETP gene that is associated with the risk of developing or expediting a CAD, wherein the at least one SNP comprises: rs3764261 [SEQ ID NO:33]

In certain embodiments, the kit comprises at least one of the following components: one or more primers for amplifying at one or more SNPs of the CETP gene; at least one enzyme for nucleic acid amplification; at least one enzyme suitable for performing an analysis for identifying at least one risk marker; and instructions for use.

In another broad aspect, there is provided herein a diagnostic kit for detecting one or more coronary artery disease (CAD)-associated polymorphisms in a genetic sample, comprising at least one probe for assessing the presence of a single nucleotide polymorphism (SNP): rs3764261 [SEQ ID NO:33] in the cholesteryl ester transfer protein (CETP) gene.

The diagnostic kit of the preceding claim, wherein the at least one probe is selected from the group of sense, anti-sense, and naturally occurring mutants, of rs3764261 [SEQ ID NO:33].

In certain embodiments, at least one probe being from 3 to 101 nucleotides in length.

In certain embodiments, at least one probe being a length selected from the group of from about 5 to 101, from about 7 to 101, from about 9 to 101, from about 15 to 101, from about 20 to 101, from about 25 to 101, from about 30 to 101, from about 40 to 101, from about 50 to 101, from about 60 to 101, from about 70 to 101, from about 80 to 101, from about 90 to 101, and from about 99 to 101 nucleotides in length.

In another broad aspect, there is provided herein a microarray for detecting one or more coronary artery disease (CAD)-associated polymorphisms in a genetic sample, comprising at least one probe for assessing the presence of a single nucleotide polymorphism (SNP) rs3764261 [SEQ ID NO:33] in the cholesteryl ester transfer protein (CETP) gene.

In another broad aspect, the method comprising:

analyzing a sample from a subject with a SNP detection assay to determine that the subject is homozygous for the polymorphism SNP rs3764621 in the cholesteryl ester transfer protein (CETP) gene, thereby generating a genetic analysis result;

inputting the genetic analysis result into a system, wherein the system comprises:

i) a computer processor for receiving, processing, and communicating data,

ii) a storage component for storing data which contains a reference genetic database of results of at least one genetic analysis of homozygosity with respect to response to statins, and

iii) a computer program, embedded within the computer processor, which is configured to process the homozygous genetic analysis result in the context of the reference database to determine, as an outcome, that the subject does not benefit from statin treatment;

c) processing the homozygous genetic analysis result with the computer program in the context of the reference database to determine, as an outcome, that the subject does not benefit from statin treatment;

d) communicating the outcome from the computer program; and

e) administering to the subject a treatment other than a statin treatment

In another broad aspect, there is provided herein a method of determining the need for treatment with a statin, comprising the step of identifying single nucleotide polymorphism (SNP) rs3764261 [SEQ ID NO:33] in the cholesteryl ester transfer protein (CETP) gene, in a nucleic acid sample from a subject, wherein the presence of the SNP is an indication that the subject does not have a need for the statin treatment.

Various objects and advantages of this invention will become apparent to those skilled in the art from the following detailed description of the preferred embodiment, when read in light of the accompanying drawings

### BRIEF DESCRIPTION OF THE DRAWINGS

The patent or application file contains one or more drawings executed in color and/or one or more photographs. Copies of this patent or patent application publication with color drawing(s) and/or photograph(s) will be provided by the Patent Office upon request and payment of the necessary fee.

FIG. 1A. Association of each SNP with RNA absolute allelic ratios, as measured using rs5882 [SEQ ID NO:41] as an indicator. *Since rs5882 [SEQ ID NO:41] is used as the indicator in the assay, the p value is not applicable. The allelic mRNA ratios were normalized to the overall mean allelic gDNA ratios (there was no indication of a gene dosage effect requiring normalization to the gDNA for each subject). The data are mean r S.D. (n=3-6). Rs247616 [SEQ ID NO:31] has the lowest p value, indicating it affects mRNA expression (transcription).

FIG. 1B. Log 2 AEI absolute values in rs247616 [SEQ ID NO:31] genotypes. There would not be a detectable difference in allelic expression of homozygous samples if the SNP is functional.

FIG. 2A. Association p values assessing relationship between CETP SNPs and Δ9 splice variant formation in human livers (n=94). Only rs9930761 [SEQ ID NO:35] and rs5883 [SEQ ID NO:34] in exon 8-10 region can account for increased formation of the Δ9 splice variant. rs289714 [SEQ ID NO:42] (intron 9), rs5882 [SEQ ID NO:41] (I1405V), and rs1801706 [SEQ ID NO:43] (G84A) show varying degrees of LD with rs9930761 [SEQ ID NO:35], accounting for the observed but lower association p values. Details for the SNPs are provided in FIG. 19—Table 5.

FIG. 2B. Percent Δ9 splice variant of total CETP mRNA as a function of rs5883 [SEQ ID NO:34]T>C and rs9930761 [SEQ ID NO:35]C>T. Homozygous minor allele carriers for rs5883 [SEQ ID NO:34]/rs9930761 [SEQ ID NO:35] were not observed. All livers were heterozygous for both rs9930761 [SEQ ID NO:35] and rs5883 [SEQ ID NO:34], except for two livers heterozygous only for rs9930761 [SEQ ID NO:35] which had relatively low Δ9 splice variant formation (18%), indicating that rs5883 [SEQ ID NO:34] is necessary for enhanced splicing.

FIG. 3. Schematics of the genomic CETP region spanning exons 8-10. The exonic enhancer site (ESE) in exon 9 is disrupted by rs5883 [SEQ ID NO:34]. Splice site sequences and the predicted splice branch point with rs9930761 [SEQ ID NO:35] are also depicted.

FIG. 4. CETP Gene structure, including locations of the main CETP polymorphisms.

FIGS. 5A-5B. Mfold RNA folding predictions of CETP exon 9. The rs5883 [SEQ ID NO:34] T variant (FIG. 5B) influences internal base pairing of the exon. This causes changes in nucleotide access at both 5′ and 3′ ends of the exon. FIG. 5A discloses [SEQ ID NO:8]. FIG. 5B discloses [SEQ ID NO:9].

FIG. 6. Allelic expression and splice assay standard curves. Plasmid DNA containing either the A or G allele of 1405V, or the normal (long splice) or Δ9 (short splice) isoform of CETP was diluted over 3 orders of magnitude. Allele specific or splice specific primers were used to amplify and quantitate the samples via Real-Time PCR in SYBR Green Master Mix (Applied Biosystems). Each point represents the average of 3 standard curves.

FIGS. 7A-7B. Correlation between Real-Time and fluorescent assay results. Correlation between allele specific Real Time PCR (RT) assays and SNaPshot Primer Extension (PE) assays (FIG. 7A), or splice specific Real-Time PCR (RT) assays and fluorescent splice specific primer assays (FIG. 7B) performed on the ABI 3730. Nine samples were analyzed in duplicate.

FIG. 8. An LD structure map showing r² and D′ values between all 13 SNPs.

FIG. 9. CETP rs9930761 [SEQ ID NO:35] RE assay. FIG. 9 discloses the “CETP” rs9930761 RE assay sequence as nucleotides 1-400 of SEQ ID NO: 12, as well as the forward primer, reverse primer and primer dimer sequences as SEQ ID NOs: 10, 11, 10, 11, 10 and 11, respectively, in order of appearance.

FIG. 10. CETP partial intron 8-exon 9 sequence [SEQ ID NO:12].

FIG. 11. rs9930761 [SEQ ID NO:35] assay restriction cut enzyme sequences [SEQ ID NOs:12-14] and rs9930761 [SEQ ID NO:35] primers [SEQ ID NOs:10-11].

FIG. 12. rs5883 [SEQ ID NO:34] Applied Biosystems TaqMan Assay.

FIG. 13. Context Sequence ([VIC/FAM]) [SEQ ID NOs:15].

FIG. 14. CETP rs5883 [SEQ ID NO:34] SNaPshot primer extension assay primers [SEQ ID NOs:16-20].

FIG. 15—Table 1. Association analysis of HDL-C levels with rs9930761 [SEQ ID NO:35]T>C, rs5883 [SEQ ID NO:34]C>T, and rs247616 [SEQ ID NO:31]C>T, with and without adjusting for upstream SNP rs247616 [SEQ ID NO:31]C>T, in the Whitehall II Study. Minor allele frequency (MAF) was 6.7% for rs9930761 [SEQ ID NO:35], 5.5% for rs5883 [SEQ ID NO:34], and 33.6% for rs247616 [SEQ ID NO:31]. Linkage disequilibrium (LD) for rs247616 [SEQ ID NO:31]-rs9930761 [SEQ ID NO:35] is R²=0.035 and D′=0.962 (haplotype frequencies are (versus expected under linkage equilibrium) TC 0.001 (0.023), CC 0.067 (0.044), TT 0.337 (0.315), and CT 0.595 (0.618). Note that the minor alleles rarely occur together (TC 0.001), indicating a strong negative LD between rs9930761 [SEQ ID NO:35]/rs5883 [SEQ ID NO:34] and rs247616 [SEQ ID NO:31], requiring HDL-C versus rs9930761 [SEQ ID NO:35]/rs5883 [SEQ ID NO:34] analysis conditional on rs247616 [SEQ ID NO:31]. For all SNPs see FIG. 20A—Table 6A.

FIG. 16—Table 2: Associations between CETP rs9930761 [SEQ ID NO:35] and rs5883 [SEQ ID NO:34] minor variant carriers versus homozygous wild-type carriers, sex, race, and primary event (myocardial infarction, stroke or death) in INVEST. Odds ratios represent occurrence of primary event for rs9930761 [SEQ ID NO:35]/rs5883 [SEQ ID NO:34] carriers versus homozygous wild-type carriers (for all SNPs see FIG. 21A—Table 5A).

FIG. 17—Table 3. PCR primers and amplicons for rs993076 [SEQ ID NO:46] and the CETP splice variant [SEQ ID NOs:21-23, 10-11, 16-17 and 24-30, respectively, in order of appearance]. rs5883 [SEQ ID NO:34] was determined using preselected commercial TaqMan and PCR probes (Life Technologies, Foster City, Calif.).

FIG. 18—Table 4. Polymorphisms in the CETP locus genotyped, and genotyping methods. An Applied Biosystems ABI 7000 instrument was used for genotyping with SNaPshot and TaqMan MGB ™ probes (Taq1B, rs5583 [SEQ ID NO:47], I405V, rs1800774 [SEQ ID NO:48] (Intron12), and G84A).

rs9935061 [SEQ ID NO:49] was genotyped with a BsoBI restriction enzyme assay. Primers were designed using Primer Express version 2.0 (all genotyping reagents: Life Technologies, Foster City, Calif.).

rs173539 [SEQ ID NO:32] was genotyped with a HaeIII restriction enzyme assay. The HEX labeled forward primer sequence used is-CCTGTGGTCCCAGTTACTTAGGA [SEQ ID NO: 1]. The reverse primer is CCCCAATCTGTAGTCTTTGCCA [SEQ ID NO:2].

rs247616 [SEQ ID NO:31] was genotyped using a Taq1 restriction enzyme assay. The FAM labeled forward primer is GACTCAACAACAGGGCCACA [SEQ ID NO:3]. The reverse primer is ACTTCGATTAAAAGAGTTCTGGAGATGGGTT [SEQ ID NO:4].

rs3764261 [SEQ ID NO:33] was genotyped using the GC clamp method. Forward allele specific primers used were CGTCCCGCGCCGCCCCTGTCGGTAGGCATCTTGG [SEQ ID NO:5] (Tm 91.7° C.) specific for the G allele and ACCTGTCGGTAGGCATCAGGT [SEQ ID NO:6] (Tm 66.8° C.) specific for the T allele. A common reverse primer (CAGGGCAATCAAGGCATCC) [SEQ ID NO:7] was used.

FIG. 19—Table 5. Estimated CETP haplotypes constructed from 5 SNPs genotyped in 44 liver samples (calculated with HelixTree). rs5883 [SEQ ID NO:34] and rs9930761 [SEQ ID NO:35] are in complete LD in the liver samples tested for all SNPs in these tissues. The EM probability represents ambiguity in calling the subject haplotypes. Most tissues carrying the minor T allele of rs5883 [SEQ ID NO:34] yielded unambiguous or high confidence haplotypes, showing that the minor T allele of rs5883 [SEQ ID NO:34] is predominantly associated with the major alleles of rs173539 [SEQ ID NO:32] and Taq1B, but with the minor alleles of I1405V and G84A (haplotype C_G_C_G_A). The single exception being assigned C_A_T_G_A has low EM probability so that the phasing cannot be determined with confidence.

FIG. 20A—Table 6A. Association p values between CETP SNPs and log-transformed HDL-C levels in the Whitehall II study (4,744 subjects, males plus females). SNPs with available “rs” id number and unadjusted p value <0.001 were included. Promoter/enhancer SNP rs247616 [SEQ ID NO:31] and splice SNP rs9930761 [SEQ ID NO:35] are highlighted in red. The SNP shown in blue is a representative SNPs in high LD with other promoter/enhancer SNPs, and with strong association to log-transformed HDL, also highlighted in the Dutch case-control statin study and in INVES-GENES. SNPs are presented in the order of their P values. SNPs were genotyped using Illumina (Illumina Inc. San Diego, Calif., USA) IBC Candidate Gene array, version 2 (WHII) or version 3 (Utrecht Cardiovascular Pharmacogenetics (IJCP) cohort and INVEST-GENES)**

FIG. 20B—Table 6B. Mean HDL-C levels grouped by genotype for rs247616 [SEQ ID NO:31] and rs5883 [SEQ ID NO:34] in all subjects. Because of the relatively low allele frequency of rs5883 [SEQ ID NO:34], some of the subgroups do not contain a sufficient number of subjects to obtain reliable mean HDL-C levels (e.g., the CC/TT haplotype (n=3). However, carriers heterozygous for both SNPs (TC/TC; n=142) show a substantial increase in HDL-C. In the vast majority of these subjects, the minor T allele of the two SNPs will be on opposite haplotypes.

FIG. 20C—Table 6C. Significant interaction between effects of rs5883 [SEQ ID NO:34] and rs247616 [SEQ ID NO:31] on HDL-C levels.

FIG. 21A—Table 7A. Allele associations between CETP polymorphisms and primary outcomes in the INVEST-GENES study (464 males and 402 females, all Caucasians) using an additive model. The SNPs in this table are sorted by chromosomal location. The p values are unadjusted. SNPs are presented in the order they appear in the genomic sequence.

FIG. 21B—Table 7B. Minor allele frequencies and LD between rs9930761 [SEQ ID NO:35] and rs5883 [SEQ ID NO:34] in the INVEST cohort.

FIG. 22—Table 8. CEU: Central European; JPT/HCB: Japanese/Han Chinese; YRI: Yoruban; CEU: Caucasians; AA: African Americans**PF: Pfizer study (Submitter: Albert B. Seymour, PhD, Pfizer Global R&D, Pharmacogenomics, 1 Eastern Point Road, MS_{—}8118D-3006, Groton, Conn. 06340) ***>90 years old; ****low: =/<30 mg/dl CEU and =/<37 mg/dl AA; high: >74 mg/dl.

### DETAILED DESCRIPTION

Unless otherwise defined, all technical and scientific terms used herein have the same meaning as commonly understood by one of ordinary skill in the art to which this invention belongs. For the purposes of the present invention, the following terms are defined below:

Definitions

The terms “a,” “an,” and “the” include the plural referents unless the context clearly dictates otherwise.

The terms “cardiovascular disease/s” and/or “coronary artery disease/s (CAD)” can include any disease, disorder or pathological state or condition that involves the heart and/or blood vessels, arteries and veins. It is to be understood, as used herein “CAD” also includes the resistance to a therapy such as treatment with an agent such as one or more statins.

Examples of such CAD and disorders include, but are not limited to, arterial disease, atheroma, atherosclerosis, arteriosclerosis, coronary artery disease, arrhythmia, angina pectoris, congestive heart disease, myocardial infarction, stroke, transient ischemic attack (TIA), aortic aneurysm, cardiopericarditis, infection and/or inflammation of these tissues and/or organs, as well as valvular, vascular and clotting problems, insufficiencies and/or disorders, etc.

The term “linked” can describe a region of a chromosome that is shared more frequently in family members affected by a particular disease or disorder, than would be expected or observed by chance, thereby indicating that the gene or genes or other identified marker/s within the linked chromosome region contain or are associated with an allele that is correlated with the presence of, or increased or decreased risk of the disease or disorder. Once linkage is established, association studies (linkage disequilibrium) can be used to narrow the region of interest or to identify the marker correlated with the disease or disorder.

The term “genetic marker/s” generally refers to a region of a nucleotide sequence (e.g., in a chromosome) that is subject to variability (i.e., the region can be polymorphic for a variety of alleles). For example, a single nucleotide polymorphism (SNP) in a nucleotide sequence is a genetic marker that is polymorphic for two alleles. Other examples of genetic markers can include but are not limited to microsatellites, restriction fragment length polymorphisms (RFLPs), repeats (i.e., duplications), insertions, deletions, etc.

The term “subject/s” generally refers to any animal that is susceptible to cardiovascular disease as defined herein and can include mammals, birds and reptiles. Examples of subjects can include, but are not limited to, humans, non-human primates, dogs, cats, horses, cows, goats, guinea pigs, mice, rats and rabbits, as well as any other domestic or commercially valuable animal including animal models of cardiovascular disease.

The term/s “nucleic acid/s” generally encompass both RNA and DNA, including cDNA, genomic DNA, mRNA, synthetic (e.g., chemically synthesized) DNA and chimeras of RNA and DNA. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

The term/s “isolated nucleic acid/s” generally encompass a DNA or RNA that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “isolated” generally refers to a nucleic acid or polypeptide that is substantially free of cellular material, viral material, or culture medium (when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

The term “oligonucleotide/s” generally refers to a nucleic acid sequence of at least about 6 nucleotides to about 100 nucleotides, for example, about 15 to 30 nucleotides, or about 20 to 25 nucleotides, which can be used, for example, as a primer in a PCR amplification or as a probe in a hybridization assay or in a microarray. Oligonucleotides can be natural or synthetic, e.g., DNA, RNA, modified backbones, etc. In addition, fragments or oligonucleotides of the nucleic acids can be used as primers or probes. Thus, in some embodiments, a fragment or oligonucleotide can be a nucleotide sequence that is at least 10, 15, 20, 25, 30, 35, 40, 45, 50, 55, 60, 65, 70, 75, 80, 85, 90, 100, 125, 150, 175, 200, 250, 300, 350, 400, 450, 500, 550, 600, 650, 700, 750, 800, 850, 900, 1000, 1500, 2000, 2500 or 3000 contiguous nucleotides of the nucleotide sequence of the gene of interest. Such fragments or oligonucleotides can be detectably labeled or modified, for example, to include and/or incorporate a restriction enzyme cleavage site when employed as a primer in an amplification (e.g., PCR) assay.

The terms “probe/s” or “primer/s” generally refers to single-stranded nucleic acid sequences that are complementary to a desired target nucleic acid. The 5′ and 3′ regions flanking the target complement sequence reversibly interact by means of either complementary nucleic acid sequences or by attached members of another affinity pair. Hybridization can occur in a base-specific manner where the primer or probe sequence is not required to be perfectly complementary to all of the sequences of a template. Hence, non-complementary bases or modified bases can be interspersed into the primer or probe, provided that base substitutions do not inhibit hybridization. The nucleic acid template may also include “nonspecific priming sequences” or “nonspecific sequences” to which the primers or probes have varying degrees of complementarity. In certain embodiments, a probe or primer comprises 101 or fewer nucleotides, from about 3 to 101 nucleotides, from about 5 to 85, from about 6 to 75, from about 7 to 60, from about 8 to 50, from about 10 to 45, from about 12 to 30, from about 12 to 25, from about 15 to 20, or from about any number of base pairs flanking the 5′ and 3′ side of a region of interest to sufficiently identify, or result in hybridization.

Further, the ranges can be chosen from group A and B where for A: the probe or primer is greater than 5, greater than 10, greater than 15, greater than 20, greater than 25, greater than 30, greater than 40, greater than 50, greater than 60, greater than 70, greater than 80, greater than 90 and greater than 100 base pairs in length. For B, the probe or primer is less than 102, less than 95, less than 90, less than 85, less than 80, less than 75, less than 70, less than 65, less than 60, less than 55, less than 50, less than 45, less than 40, less than 35, less than 30, less than 25, less than 20, less than 15, or less than 10 base pairs in length.

In other embodiments, the probe or primer is at least 70% identical to the contiguous nucleic acid sequence or to the complement of the contiguous nucleotide sequence, for example, at least 80% identical, at least 90% identical, at least 95% identical, and is capable of selectively hybridizing to the contiguous nucleic acid sequence or to the complement of the contiguous nucleotide sequence. In certain embodiments, preferred primer lengths include 25 to 35, 18 to 30, and 17 to 24 nucleotides. Often, the probe or primer further comprises a label, e.g., a radioisotope, fluorescent compound, enzyme, or enzyme co-factor.

To obtain high quality primes, primer length, melting temperature (Tₘ), GC content, specificity, and intra- or inter-primer homology are taken into account.

The probes or primers can also be variously referred to as antisense nucleic acid molecules, polynucleotides or oligonucleotides, and can be constructed using chemical synthesis and enzymatic ligation reactions known in the art. For example, an antisense nucleic acid molecule (e.g. an antisense oligonucleotide) can be chemically synthesized using naturally occurring nucleotides or variously modified nucleotides designed to increase the biological stability of the molecules or to increase the physical stability of the duplex formed between the antisense and sense nucleic acids. The primers or probes can further be used in Polymerase Chain Reaction (“PCR”) amplification.

The term “genetic material/s” generally refers to a nucleic acid sequence that is sought to be obtained from any number of sources, including without limitation, whole blood, a tissue biopsy, lymph, bone marrow, hair, skin, saliva, buccal swabs, purified samples generally, cultured cells, and lysed cells, and can comprise any number of different compositional components (e.g. DNA, RNA, tRNA, siRNA, mRNA, or various non-coding RNAs). The nucleic acid can be isolated from samples using any of a variety of procedures known in the art. In general, the target nucleic acid will be single stranded, though in some embodiments the nucleic acid can be double stranded, and a single strand can result from denaturation. It will be appreciated that either strand of a double-stranded molecule can serve as a target nucleic acid to be obtained. The nucleic acid sequence can be methylated, non-methylated, or both, and can contain any number of modifications. Further, the nucleic acid sequence can refer to amplification products as well as to the native sequences.

The terms “heterozygous” or “heterozygous polymorphism” generally refers to the two alleles of a diploid cell or organism at a given locus are different, that is, that they have a different nucleotide exchanged for the same nucleotide at the same place in their sequences.

The terms “homozygous” or “homozygous polymorphism” generally refer to the two alleles of a diploid cell or organism at a given locus are identical, that is, that they have the same nucleotide for nucleotide exchange at the same place in their sequences.

The terms “hybridization” or “hybridizing,” generally refer to the formation of A-T and C-G base pairs between the nucleotide sequence of a fragment of a segment of a polynucleotide and a complementary nucleotide sequence of an oligonucleotide. By complementary is meant that at the locus of each A, C, G or T (or U in a ribonucleotide) in the fragment sequence, the oligonucleotide sequenced has a T, G, C or A, respectively. The hybridized fragment/oligonucleotide is called a “duplex.” A “hybridization complex”, such as in a sandwich assay, means a complex of nucleic acid molecules including at least the target nucleic acid and a sensor probe. It may also include an anchor probe.

The terms “locus” or “loci” generally refer to the site of a gene on a chromosome. Pairs of genes, known as “alleles” control the hereditary trait produced by a gene locus. Each animal's particular combination of alleles is referred to as its “genotype”. Where both alleles are identical the subject is said to be homozygous for the trait controlled by that gene pair; where the alleles are different, the subject is said to be heterozygous for the trait.

The term “melting temperature” generally refers to the temperature at which hybridized duplexes dehybridize and return to their single-stranded state. Likewise, hybridization will not occur in the first place between two oligonucleotides, or, herein, an oligonucleotide and a fragment, at temperatures above the melting temperature of the resulting duplex.

General Description

Polymorphisms in Cholesteryl Ester Transfer Protein (CETP) influences CETP activity, and are thought to affect risk for coronary artery disease (CAD), and treatment outcome with statin drugs, but the causative CETP variants remained unclear. A major CETP splice isoforms (referred to here as ‘CETP splice variant’) containing an in-frame deletion of exon 9 dimerizes with the full-length protein, preventing secretion in a dominant-negative manner, again with unknown in vivo consequences.

Genetic markers are non-invasive, cost-effective and conducive to mass screening of subjects. The SNPs identified herein can be effectively used alone or in combination with other SNPs as well as with other clinical markers for risk-stratification, assessment, and diagnosis of non-response to cardiac medications. Further, these genetic markers in combination with other clinical markers for CAD. The genetic markers taught herein provide greater specificity and sensitivity in identification of subjects at risk for CAD due to non-response to cardiac medications.

To search for regulatory variants affecting CETP mRNA expression and test for the presence of genetic effects on splicing, the inventors measured allelic mRNA expression and splicing in human livers, identifying candidate promoter/enhancer SNPs located 2.5-7 kb upstream, and discovering two SNPs in near complete linkage disequilibrium (LD) tightly associated with '9 CETP splicing. The inventors determined whether these polymorphisms affect HDL-C and risk for myocardial infarction, in healthy subjects, those already with risk factors, and effectiveness of statins to prevent CAD.

The present invention is based, at least in part, on the inventors' discovery of a correlation between genetic markers located on the long (q) arm of chromosome 16 at position 21 (more precisely, the CETP gene is located from base pair 56,995,834 to base pair 57,017,755 on chromosome 16), and various aspects of cardiovascular disease and treatment. Thus, in one aspect, there is provided herein a method of identifying a subject having either an increased or decreased risk of developing cardiovascular disease, comprising detecting in the subject one or more genetic markers in the CETP gene correlated with an increased or decreased risk of developing cardiovascular disease, or responding to statins. Using these CETP variants, those skilled in the art will also be able to apply the CETP variants in testing effects on the response to the new drug class of CETP inhibitors.

Further provided is a method of identifying a subject having either an increased or decreased risk of developing cardiovascular disease, comprising: a) correlating the presence of one or more genetic markers in the CETP gene with an increased or decreased risk of developing cardiovascular disease; and b) detecting the one or more genetic markers of step (a) in the subject, thereby identifying the subject as having an increased or decreased risk of developing cardiovascular disease.

In further embodiments, there is provided herein a method of correlating a genetic marker in the CETP gene with an increased risk of developing cardiovascular disease, comprising: a) detecting in a subject with cardiovascular disease the presence of one or more genetic markers in the CETP gene; and b) correlating the presence of the one or more genetic markers of step (a) with cardiovascular disease in the subject.

Also provided is a method of correlating a genetic marker in the CETP gene with a decreased risk of developing cardiovascular disease, comprising: a) detecting in a subject without cardiovascular disease the presence of one or more genetic markers in the CETP gene; and b) correlating the presence of the one or more genetic markers of step (a) with the absence of cardiovascular disease in the subject.

Additionally provided herein is a method of diagnosing cardiovascular disease in a subject, comprising detecting in the subject one or more genetic markers correlated with a diagnosis of cardiovascular disease, as well as a method of diagnosing cardiovascular disease in a subject, comprising: a) correlating the presence of one or more genetic markers in the CETP gene with a diagnosis of cardiovascular disease; and b) detecting the one or more genetic markers of step (a) in the subject, thereby diagnosing cardiovascular disease in the subject.

A method is also provided of correlating a genetic marker in the CETP gene with a diagnosis of cardiovascular disease, comprising: a) detecting in a subject diagnosed with cardiovascular disease the presence of one or genetic markers in the CETP gene; and b) correlating the presence of the one or more genetic markers of step (a) with a diagnosis of cardiovascular disease in a subject.

In the methods described herein, the detection of a genetic marker in a subject can be carried out according to methods well known in the art. For example DNA is obtained from any suitable sample from the subject that will contain DNA and the DNA is then prepared and analyzed according to well-established protocols for the presence of genetic markers according to the methods described herein. In some embodiments, analysis of the DNA can be carried by amplification of the region of interest according to amplification protocols well known in the art (e.g., polymerase chain reaction, ligase chain reaction, strand displacement amplification, transcription-based amplification, self-sustained sequence replication (3SR), Q.beta. replicase protocols, nucleic acid sequence-based amplification (NASBA), repair chain reaction (RCR) and boomerang DNA amplification (BDA)). The amplification product can then be visualized directly in a gel by staining or the product can be detected by hybridization with a detectable probe. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, or by electrophoresis. Thus, there is also provided herein oligonucleotides for use as primers and/or probes for detecting and/or identifying genetic markers according to the methods described herein.

The genetic markers are correlated with various aspects of cardiovascular disease as described herein according to methods disclosed in the Examples provided herein for correlating genetic markers with various phenotypic traits, including disease states and pathological conditions and levels of risk associated with developing a disease or pathological condition. In general, identifying such correlation involves conducting analyses that establish a statistically significant association and/or a statistically significant correlation between the presence of a genetic marker or a combination of markers and the phenotypic trait in the subject. An analysis that identifies a statistical association (e.g., a significant association) between the marker or combination of markers and the phenotype establishes a correlation between the presence of the marker or combination of markers in a subject and the particular phenotype being analyzed. The correlation can involve one or more than one genetic marker (e.g., two, three, four, five, or more) in any combination.

In one aspect, there is provided herein a method for determining in a subject the association between genotype(s) and coronary artery disease (CAD). It is to be understood that the use of the term CAD is meant to include one or more of the following: i) risk of developing a cardiovascular disorder; ii) drug response; and/or iii) suitability to a treatment regime.

Thus, according to one aspect there is provided a method of predicting or determining a subject's response to an agent. The method generally comprises analyzing a sample from the subject for the presence or absence of one or more polymorphisms selected from the group consisting of: determine the presence of at least one of three polymorphisms (rs5883 [SEQ ID NO:34], rs9930761 [SEQ ID NO:35] and rs247616 [SEQ ID NO:31]) in the cholesteryl ester transfer protein (CETP) gene.

In one embodiment, the presence or absence of one or more of the polymorphisms is indicative of the subject's response to the agent. In one embodiment, the presence of one or more of the polymorphisms is generally indicative of a decreased response of a subject to an agent.

In another embodiment, one or both of the following polymorphisms may be utilized in conjunction with one or more or all of the polymorphisms. In this embodiment, the presence of one or more of the polymorphisms is generally indicative of a decreased response of a subject to an agent. In another embodiment, the presence of one or more of the polymorphism can be indicative of an increased response of a subject to an agent.

In another embodiment, the presence in a subject of polymorphisms (rs5883 [SEQ ID NO:34], rs9930761 [SEQ ID NO:35] and rs247616 [SEQ ID NO:31]) will indicate an enhanced response of the subject to an increased dose, or increasing doses, of an agent. In one aspect, the subjects are non-responders or poor responders.

In any of the embodiments, the agent comprises a lipid-lowering agent such as a HMG-CoA reductase inhibitor (e.g., a statin), including but not limited to, one or more statins.

In one particular embodiment, the genotype of the subject includes C>T at position: Exon 9+121 (rs5883 [SEQ ID NO:34]) in the gene encoding CETP, alone or together with one or more or all of the above polymorphisms. The presence of one or more of these polymorphisms in a subject indicates a reduced response to statins.

The one or more polymorphisms can be detected directly or by detection of one or more polymorphisms which are in linkage disequilibrium with the one or more polymorphisms.

The methods described herein are particularly useful in subjects at risk of or suffering from a disease or condition associated with coronary artery disease (CAD), including myocardial infarctions, or subjects undergoing or who have undergone treatment for one or more of these diseases or conditions, including surgical treatment, including placement of stents, bypass surgery, valve replacement, and the like.

Where the following discussion refers to aspects useful to predict or determine a subject's response to one or more agents, it will be appreciated that these aspects are also useful in determining a subject's suitability for a treatment regime, preferably in determining a subject's suitability to prophylactic or therapeutic treatment with an agent.

It will be appreciated that the methods described herein identify several categories of polymorphisms or polymorphism combinations—namely those associated with poor response or resistance to one or more agents (referred to herein as “resistance polymorphisms”), those associated with response to one or more agents (referred to herein as “responsive polymorphisms”), those associated with response to one or more agent treatment regimens, and those associated with response to varying doses, including increased dosages, of one or more agents.

In certain embodiments, these SNPs can be aggregated into a scoring system that allows the stratification of a subject into a responder category i.e., responder, mildly reduced response, moderately reduced response or non-responder. Absence of any one of the identified resistance polymorphisms places a subject into the responder category. Therefore, there is also provided herein a method of predicting or determining a subject's response to an agent, the method comprising: determining the presence or absence of at least one resistance polymorphism associated with poor response or resistance to an agent; wherein the presence of at least one resistance polymorphism is indicative of poor response or resistance to an agent. Again, it will be appreciated that the above aspect may be used to determine a subject's suitability for a treatment regime or dosage, preferably in determining a subject's suitability to prophylactic or therapeutic treatment with an agent regime or dosage.

In one embodiment, the presence of all of the responsive polymorphisms is indicative of a response to an increased risk of CAD. In another embodiment, the presence of two or more resistance polymorphisms is indicative of poor response or resistance to an agent.

In another aspect, there is provided herein a method of predicting or determining a subject's response to an agent. The method generally comprises: providing the result of one or more genetic tests of a sample from the subject, and analyzing the result for the presence or absence of one or more polymorphisms selected from the groups described herein (or one or more polymorphisms in linkage disequilibrium with any one or more of these polymorphisms); or one or more polymorphisms in linkage disequilibrium with any one or more of these polymorphisms; wherein a result indicating the presence or absence of one or more of the polymorphisms is indicative of the subject's response to an agent.

In one embodiment, the methods as described herein are performed in conjunction with an analysis of one or more risk factors, including one or more epidemiological risk factors, associated with a response to an agent. Such epidemiological risk factors include but are not limited to smoking or exposure to tobacco smoke, age, sex, and familial history of a disease or condition associated with platelet aggregation, including coronary artery disease, acute coronary syndrome, peripheral vascular disease, atherosclerosis including symptomatic atherosclerosis, cerebrovascular diseases, thromboembolism, or subjects undergoing or who have undergone treatment for one or more of these diseases or conditions, including surgical treatment, including placement of stents, bypass surgery, valve replacement, and the like.

In another aspect there is provided herein a set of nucleotide probes and/or primers for use in the preferred methods described herein. In certain embodiments, the nucleotide probes and/or primers are those which span, or are able to be used to span, the polymorphic regions of the gene/s. Also provided are one or more nucleotide probes and/or primers comprising the sequence of any one of the probes and/or primers herein described.

In yet a further aspect, there is provided herein a nucleic acid microarray for use in these methods, which microarray comprises a substrate presenting nucleic acid sequences capable of hybridizing to nucleic acid sequences which encode one or more of the resistance or responsive polymorphisms described herein or sequences complementary thereto.

In another aspect, there is provided herein an antibody microarray for use in these methods, which microarray comprises a substrate presenting antibodies capable of binding to a product of expression of a gene the expression of which is upregulated or downregulated when associated with a resistance or responsive polymorphism as described herein.

In still a further aspect, there is described herein a method of assessing a subject's suitability for an intervention that is diagnostic of or therapeutic for a disease associated with CAD, the method comprising: a) providing the result of one or more genetic tests of a sample from the subject, and b) analyzing the result for the presence or absence of one or more responsive polymorphisms or for the presence or absence of one or more resistance polymorphisms, wherein the responsive or resistance polymorphisms (rs5883 [SEQ ID NO:34], rs9930761 [SEQ ID NO:35] and rs247616 [SEQ ID NO:31]) in the cholesteryl ester transfer protein (CETP) gene; or one or more polymorphisms which are in linkage disequilibrium with any one or more of the polymorphisms; wherein the presence of one or more resistance polymorphisms is indicative of the subject's suitability or unsuitability for the intervention.

In one embodiment, the intervention is a diagnostic test for a disease or condition associated with CAD, including acute coronary syndrome, peripheral vascular disease, atherosclerosis including symptomatic atherosclerosis, cerebrovascular diseases, thromboembolism, or subjects undergoing or who have undergone treatment for one or more of these diseases or conditions, including surgical treatment, including placement of stents, bypass surgery, valve replacement, and the like.

In another embodiment, the intervention is a therapy for a disease or condition associated with CAD, including acute coronary syndrome, peripheral vascular disease, atherosclerosis including symptomatic atherosclerosis, cerebrovascular diseases, thromboembolism, or subjects undergoing or who have undergone treatment for one or more of these diseases or conditions, including surgical treatment, including placement of stents including a determination of whether a drug eluting or bare metal coronary stent is to be implanted, bypass surgery, valve replacement, and the like, more preferably a preventative therapy for the diseases or conditions.

In a still further aspect, there is provided herein a method for the use of data predictive of the responsiveness of a subject to an agent in the determination of the subject's suitability for an intervention that is diagnostic of or therapeutic for a disease or condition associated with CAD, the data comprising, consisting of or including the result of at least one agent response-associated genetic analysis selected from one or more of the genetic analyses described herein, and the data being indicative of the subject's suitability or unsuitability for the intervention. In one embodiment, the data is representative of the presence of one or more responsive polymorphisms, or is representative of the absence of one or more responsive polymorphisms, or is representative of the presence of one or more resistance polymorphisms.

In another aspect, there is provided herein a system for determining a subject's response to an agent, the system comprising: computer processor means for receiving, processing and communicating data; storage means for storing data including a reference genetic database of the results of at least one genetic analysis with respect to response to an agent or with respect to a disease or condition associated with CAD, and optionally a reference non-genetic database of non-genetic risk factors for a disease or condition associated with CAD; and a computer program embedded within the computer processor which, once data consisting of or including the result of a genetic analysis for which data is included in the reference genetic database is received, processes the data in the context of the reference databases to determine, as an outcome, the subject's response to an agent, the outcome being communicable once known, preferably to a user having input the data. In one embodiment, the data is input by a representative of a healthcare provider. In another embodiment, the data is input by the subject, their medical advisor or other representative. Preferably, the system is accessible via the internet or by subjectal computer. Preferably, the reference genetic database consists of, comprises or includes the results of an agent response-associated genetic analysis selected from one or more of the genetic analyses described herein. The reference genetic database may additionally comprise or include the results of an analysis of one or more further polymorphisms. Also, the reference genetic database consists of, comprises or includes the results of all of the genetic analyses described herein.

In yet a further aspect, there is provided herein a computer program suitable for use in a system as defined above comprising a computer usable medium having program code embodied in the medium for causing the computer program to process received data consisting of or including the result of at least one agent response-associated genetic analysis in the context of both a reference genetic database of the results of the at least one agent response-associated genetic analysis, and, optionally, a reference non-genetic database of non-genetic risk factors for a disease or condition associated with CAD.

Also provided are computer systems and programs as described above for the determination of the subject's suitability for an intervention that is diagnostic of or therapeutic for a disease or condition associated with CAD.

In a still further aspect, there is provided herein the use of data predictive of the predisposition of a subject to a disease or condition associated with CAD in the prediction or determination of the subject's response to an agent, the data comprising, consisting of or including the result of at least one agent response-associated genetic analysis selected from one or more of the genetic analyses described herein, and the data being representative of the subject's response to an agent.

In a further aspect, there is provided herein a kit for assessing a subject's response to an agent, the kit comprising a means of analyzing a sample from the subject for the presence or absence of one or more polymorphisms disclosed herein.

In still a further aspect, there is provided herein a method of assessing a subject's suitability for a therapeutic intervention for a disease associated with CAD, the method comprising: a) providing the result of one or more tests for the presence of at least one of three single nucleotide polymorphisms (SNPs) (rs5883 [SEQ ID NO:34], rs9930761 [SEQ ID NO:35] and rs247616 [SEQ ID NO:31]) in the cholesteryl ester transfer protein (CETP) gene in a sample from the subject; and b) analyzing the result; wherein a result indicative of a minor allele of at least one SNT is indicative of the subject's suitability or unsuitability for the intervention.

In a further aspect, there is provided a method of predicting or determining the efficacy of a treatment regimen of a subject for CAD, wherein the treatment regimen comprises administration of an agent, the method comprising a) providing the result of one or more tests for the presence of one or more SNPs described herein in a sample from the subject; and b) analyzing the result; wherein a result indicative of such presence is indicative of reduced efficacy of the treatment regimen. In certain embodiments, the efficacy is long-term efficacy; including the efficacy of the treatment regimen after one week of treatment, after one month of treatment, or after between about one month and one year of treatment.

In a particular aspect, there is provided herein methods for determining that a human has an increased risk for developing cardiovascular disorders or statin resistance, comprising: testing nucleic acid from the human to determine the presence of at least one of at least one polymorphisms in a cholesteryl ester transfer protein (CETP) gene wherein the presence of a minor allele of the polymorphisms is detected and indicates that the human has an increased risk for developing cardiovascular disease, including myocardial infarction, and statin resistance.

Resistance and/or Responsive Genetic Polymorphisms

A resistance genetic polymorphism (also referred to herein as a resistance polymorphism) is one which, when present, is indicative of poor response or resistance to an agent. In contrast, a responsive genetic polymorphism (also referred to herein as a responsive polymorphism) is one which, when present, is indicative of a response to an agent.

As used herein, the term “response” when used in reference to an agent means the susceptibility of the subject to the effect(s) of an agent and refers to the efficacy of the agent in the subject. Accordingly, the phrase “a response to an agent” means that a subject having such a response possesses a hereditary inclination or tendency to respond to an agent, or that the subject exhibits a susceptibility to the effect(s) of an agent, such that the agent has efficacy. This does not necessarily mean that such a subject will always respond to a given gent at any time, merely that he or she has a greater likelihood of responding compared to the general population of subjects that either possess a polymorphism associated with poor response or resistance or do not possess a polymorphism associated with a response to an agent (referred to herein as a responsive polymorphism).

Similarly, the phrase “poor response or resistance to an agent” means that a subject having such a poor response or resistance possesses a hereditary disinclination or reduced tendency to respond to an agent, or that the subject exhibits a decreased susceptibility to the effect(s) of an agent, such that the agent has decreased or no efficacy. Accordingly, poor response or resistance includes non-response. This does not necessarily mean that such a subject will never respond to a given agent at any time, merely that he or she has a greater likelihood of not responding compared to the general population of subjects that either does not possess a polymorphism associated with poor response or resistance or does possess a polymorphism associated with a response to an agent (referred to herein as a responsive polymorphism).

A subject's response to an agent may be determined by analyzing a sample from the subject for the presence or absence of a polymorphism; or one or more polymorphisms which are in linkage disequilibrium therewith. These polymorphisms can also be analyzed in combinations of two or more, or in combination with other polymorphisms indicative of a subject's response to an agent. Assays which involve combinations of polymorphisms, including those amenable to high throughput, such as those utilizing microarrays, are within the contemplated scope of the methods described herein.

The one or more polymorphisms can be detected directly or by detection of one or more polymorphisms which are in linkage disequilibrium with the one or more polymorphisms. Linkage disequilibrium (LD) is a phenomenon in genetics whereby two or more mutations or polymorphisms are in such close genetic proximity that they are co-inherited. This means that in genotyping, detection of one polymorphism as present infers the presence of the other. (Reich D E et al; Linkage disequilibrium in the human genome, Nature 2001, 411:199-204.).

Statistical analyses show that the genetic assays can be used to determine the suitability of any subject to a treatment regime, preferably prophylactic or therapeutic treatment with an agent, and in particular to identify subjects with poor response or resistance to an agent. The analysis can be of combinations of resistance polymorphisms only, of responsive polymorphisms only, or of combinations of both. Analysis can also be step-wise, with analysis of the presence or absence of responsive polymorphisms occurring first and then with analysis of resistance polymorphisms proceeding only where no responsive polymorphisms are present. Thus, through systematic analysis of the frequency of these polymorphisms in well defined groups of subjects as described herein, it is possible to implicate certain genes and proteins in the response to an agent and improve the ability to identify which subjects have poor response or resistance to an agent, and to identify those subjects who would benefit from a particular treatment regime.

Thus, another aspect, there is provided herein methods for tailoring a subject's prophylactic or therapeutic treatment with one or more agents, preferably one or more statin agents according to that subject's drug response genotype.

Statin Agents

As used herein, the term “statin agent” and grammatical equivalents thereof refers to an agent, including a drug, compound, biologic or combination thereof, that comprises a lipid-lowering agent such as a HMG-CoA reductase inhibitor, including but not limited to, one or more statins. Statins (or HMG-CoA reductase inhibitors) are a class of drugs used to lower cholesterol levels by inhibiting the enzyme HMG-CoA reductase, which plays a central role in the production of cholesterol in the liver. Non-limiting examples of statins include: atorvastatin, such as Lipitor®, Torvast®; atorvastatin+amlodipidine such as Besylate®; cerivastatin such as Lipobay®; cholystyramine; colestipol; fluvastatin such as Lescol®, Lescol XL®; gemfibrozil; lovastatin such as Mevacor®, Altocor®, Altoprev®; Lovastatin+niacin such as extended-release Advicor®; mevastatin such as Compactin®; Pitavastatin such as Livalo®, Pitava®; pravastatin such as Pravachol®, Selektine®, Lipostat®; probucol; rosuvastatin such as Crestor®; simvastatin such as Zocor®, Lipex®; simvastatin+ezetimibe such as Vytorin®; and, simvastatin+niacin such as extended-release Simcor®.

### EXAMPLES

Certain embodiments are defined in the Examples herein. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference herein. Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

### Example 1

Total CETP mRNA Levels in Human Livers and CETP Genotype

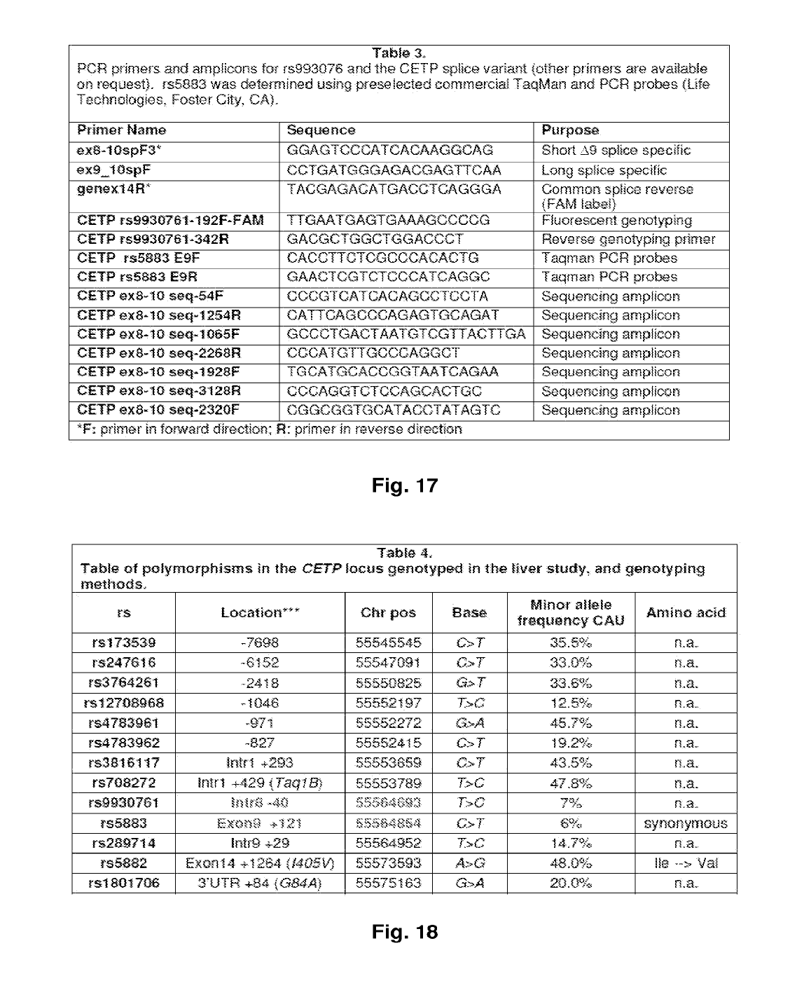

PCR cycle thresholds (CTs; mean 27.9±1.1 SD) varied considerably for CETP mRNA between tissues. To scan the CETP locus for polymorphisms associated with mRNA expression, we genotyped multiple SNPs spanning ˜37 kb (FIG. 4, FIG. 17—Table 3). None of these SNPs yielded a robust association with overall mRNA levels.

Allelic CETP mRNA Ratios in Liver

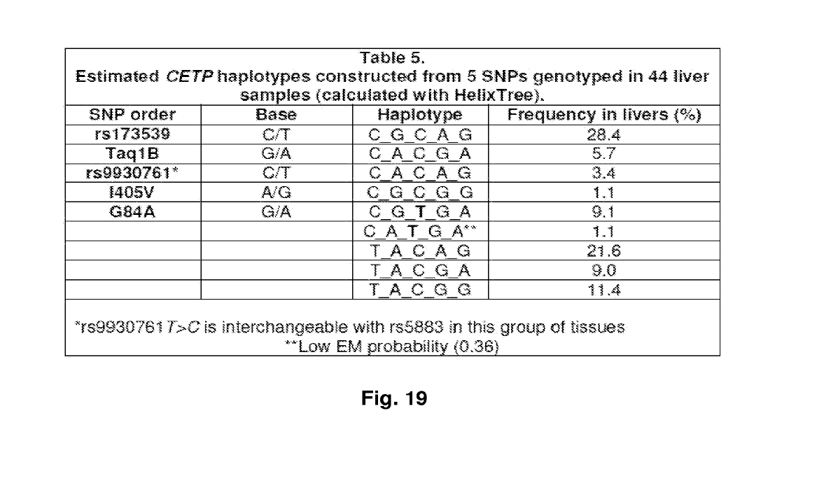

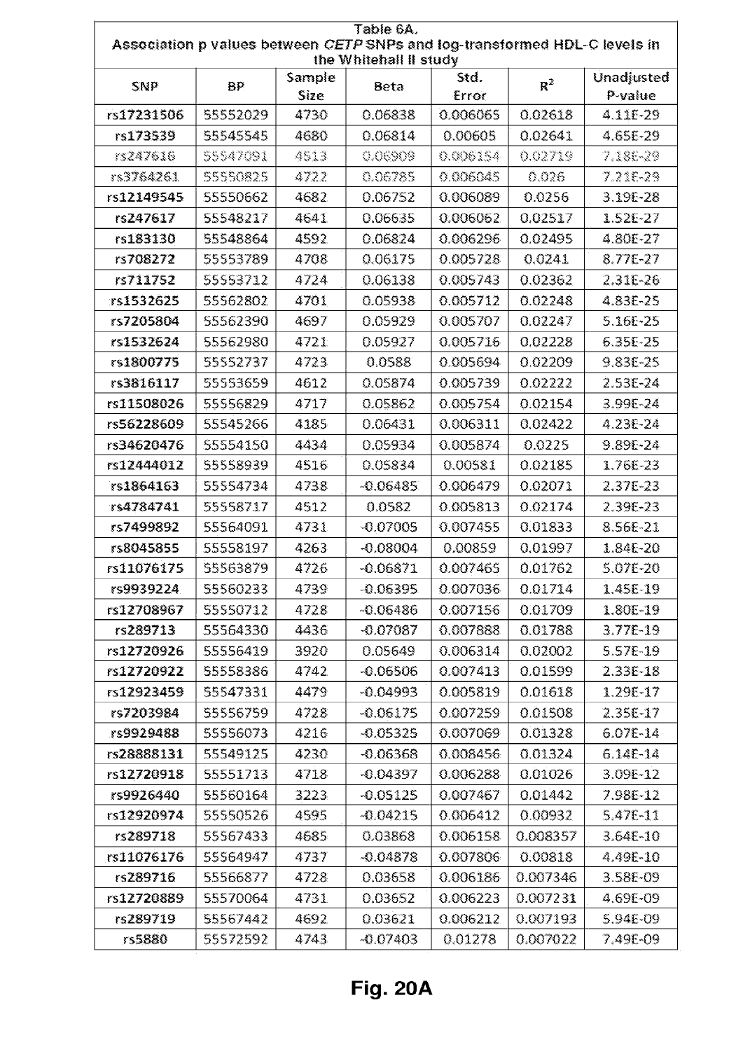

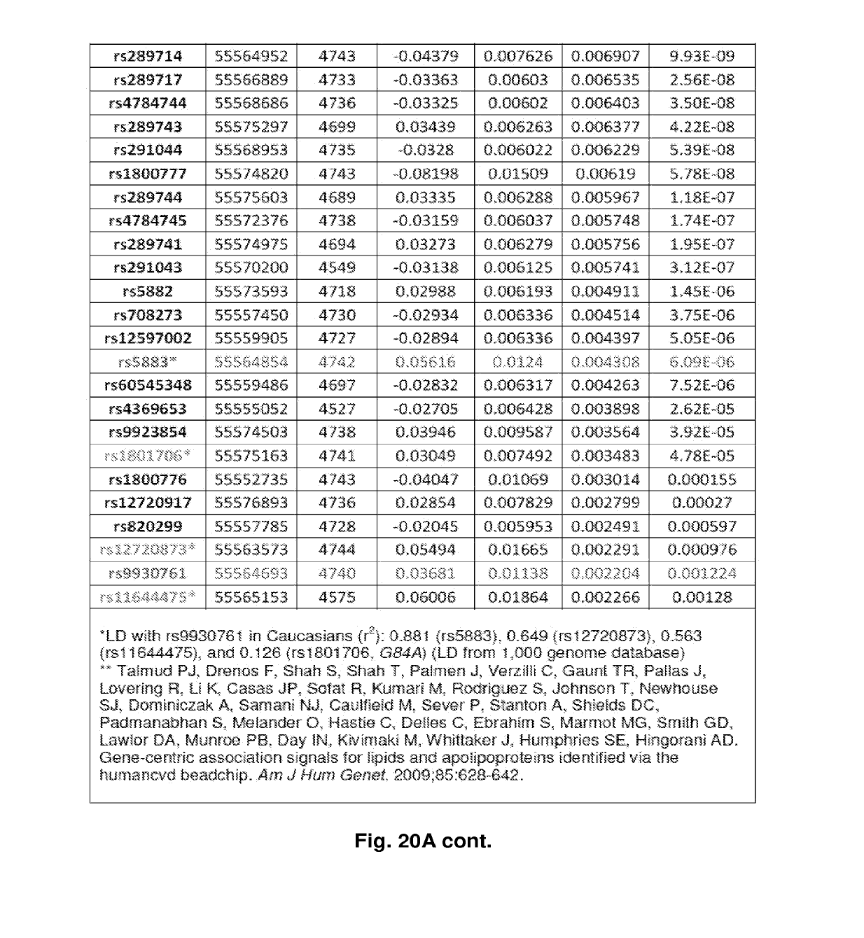

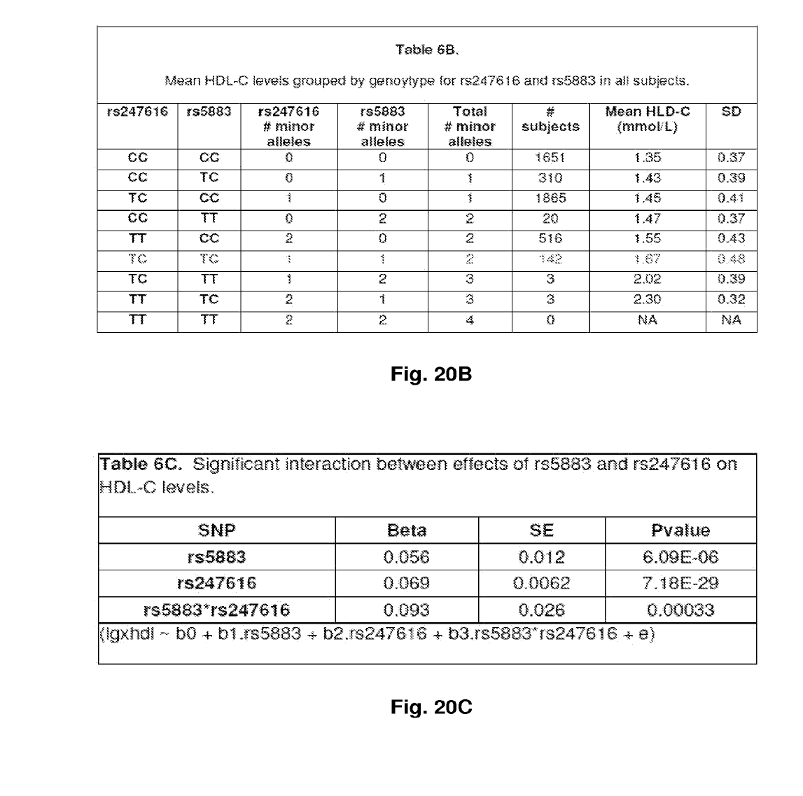

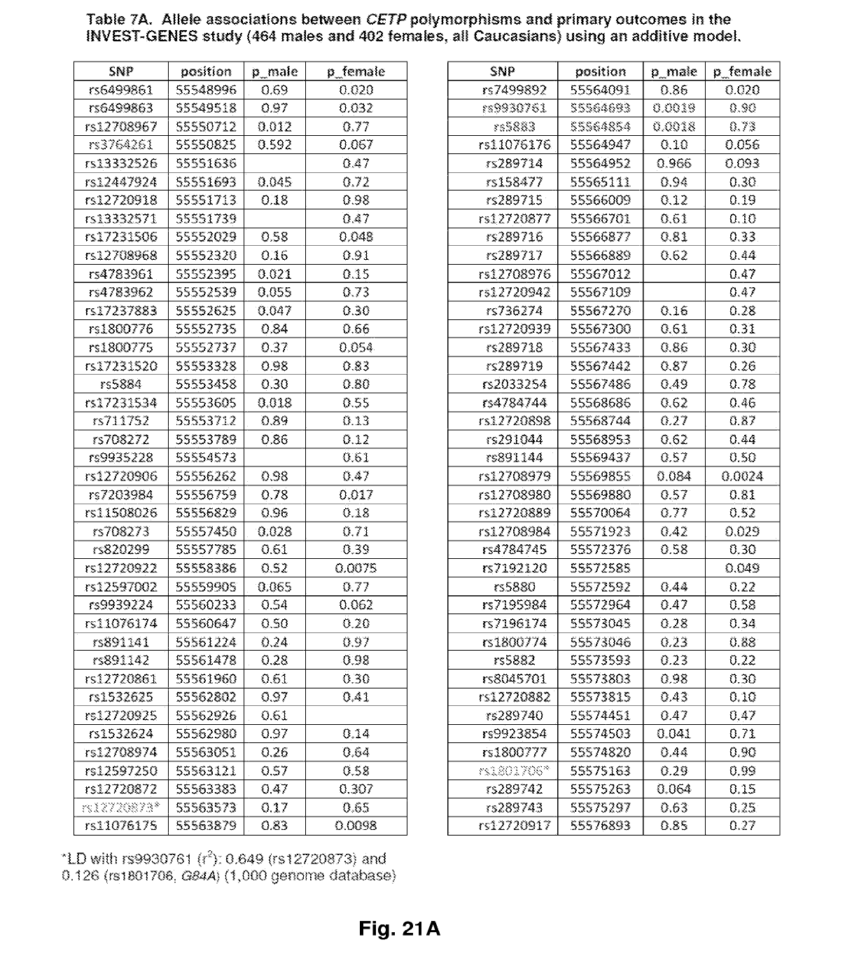

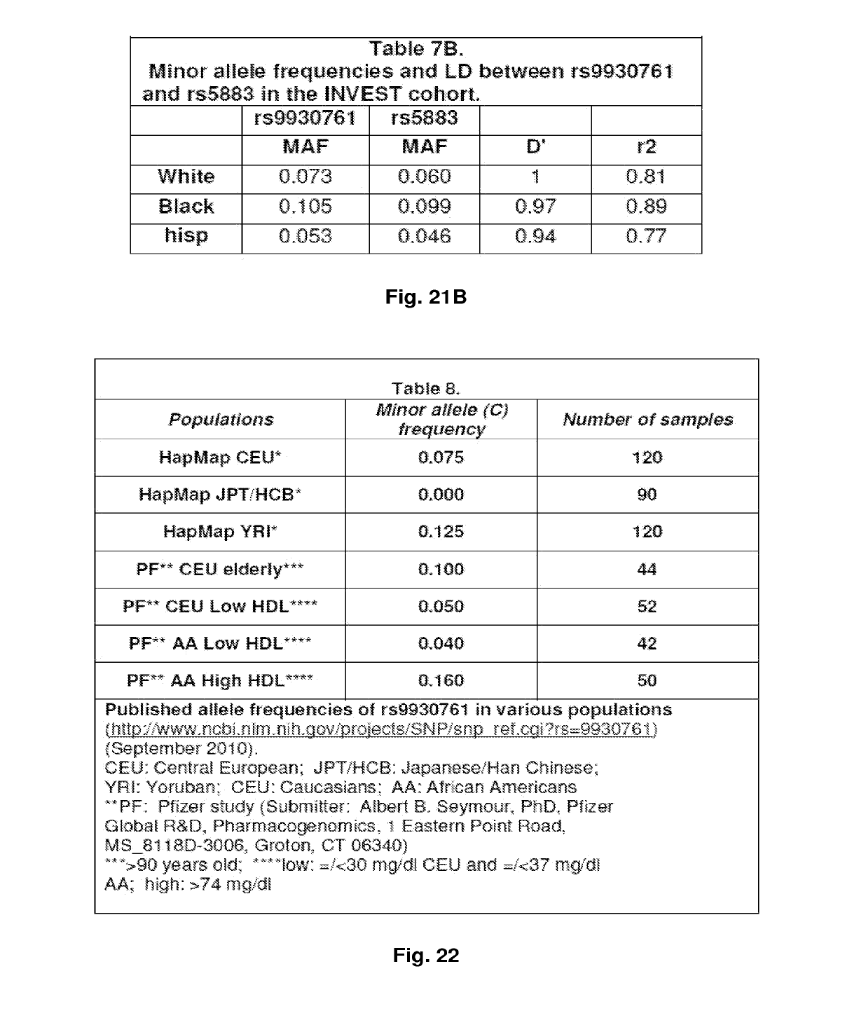

Using the SNaPshot™ primer extension assay, allelic mRNA ratios were measurable in 56 livers with rs5882 [SEQ ID NO:41] as the marker SNP. Significant allelic expression imbalance (AEI) was detectable in 29 of 56 livers tested (AEI ratios log₂>0.4, or >30% below or above the mean gDNA ratio). The allelic mRNA ratios were distributed above and below the mean DNA ratio, indicating the presence of one or more cis-acting regulatory polymorphisms present in low LD with the marker SNP rs5882 [SEQ ID NO:41]. Scanning the CETP locus with 13 SNPs genotyped in livers (FIG. 18—Table 4), the inventors discovered SNP associations with presence of absence of AEI, or AEI ratios as continuous variable (absolute log ratios only).

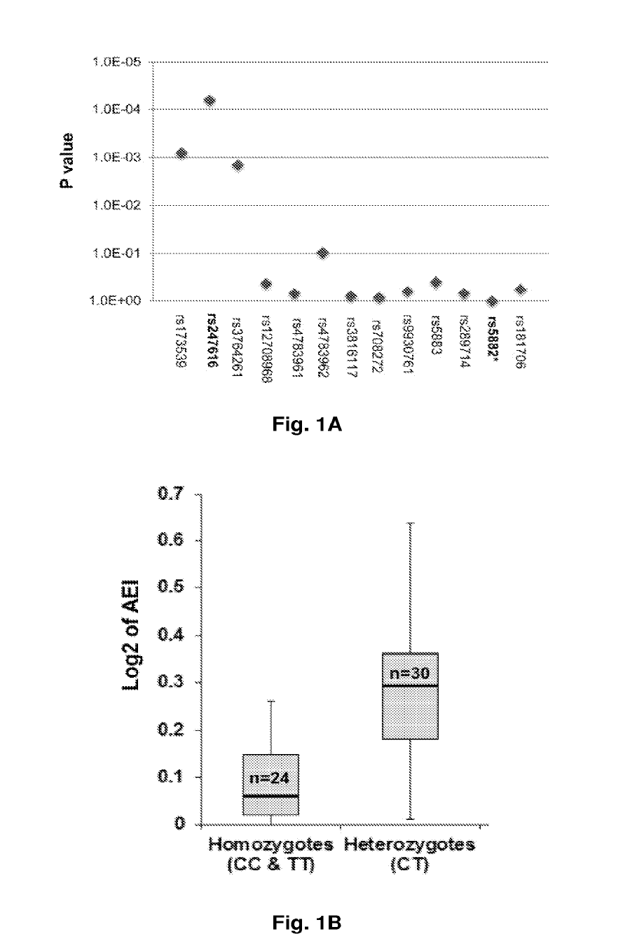

Shown in FIG. 1A, three SNPs located 2.6-7 kb upstream scored significantly (rs173539 [SEQ ID NO:32], rs247616 [SEQ ID NO:31], and rs3764261 [SEQ ID NO:33]), with rs247616 [SEQ ID NO:31] having the strongest association (p=6.4×E-5), indicating that transcription is under genetic control by these variants or others in high LD across the large 5′-haplotype block. Other promoter SNPs did not score significantly (FIG. 1A). The allelic mRNA ratios differed strongly between genotypes of rs247616 [SEQ ID NO:31] (FIG. 1B).

Association of CETP Exon 9 rs5883 C>T [SEQ ID NO:341 and Intron 8 SNP rs9930761T>C [SEQ ID NO:35] with the Δ9 CETP mRNA Splice Variant in Liver

Measured with fluorescently labeled PCR primers, the Δ9 splice variant accounted for 10% to 48% of total CETP mRNA in 94 livers analyzed. An initial CETP SNP scan first revealed an association of 1405V (rs5882 [SEQ ID NO:41]) and G84A in the 3′UTR (rs1801706 [SEQ ID NO:43]) with increased Δ9 splice variant, showing the presence of a splicing polymorphism.

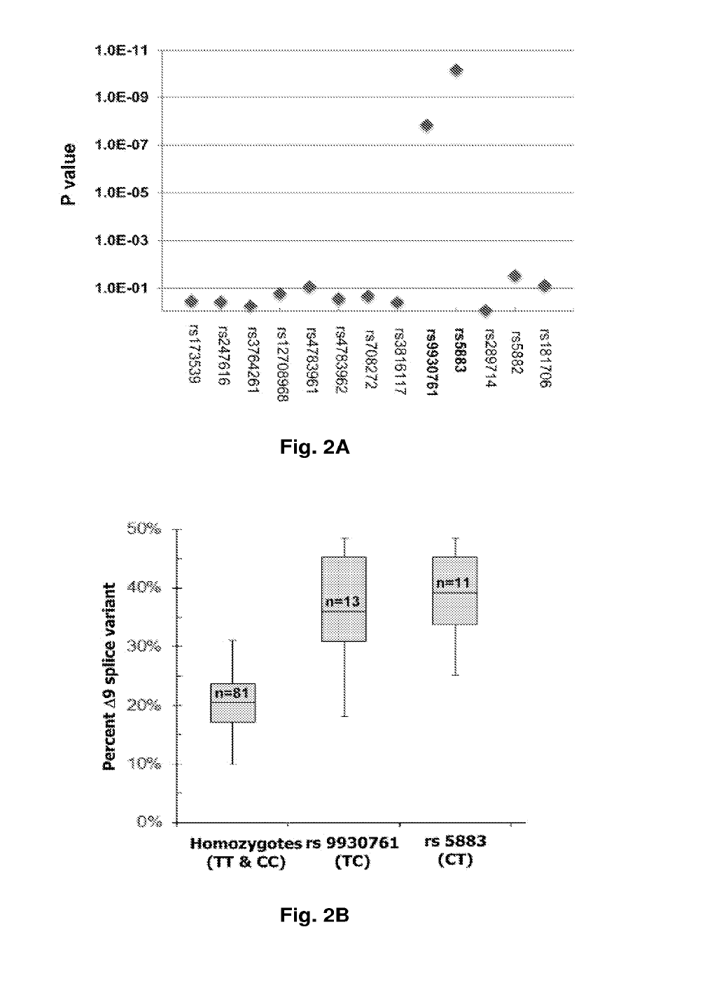

Sequencing a 3128 by CETP genomic DNA region containing exon 8 through exon 10 in 6 liver tissues with low and high Δ9 splice variant expression yielded only two SNPs, in intron 8 (rs9930761T>C [SEQ ID NO:35]) and exon 9 (rs5883 [SEQ ID NO:34]), present in all three tissues with high, and absent in those with low Δ9 expression. In 94 livers, rs5883 [SEQ ID NO:34] and rs9930761 [SEQ ID NO:35] (in complete LD (D′=1); MAF 5.9% and 6.9%%, respectively) were the only SNPs strongly associated with the Δ9 splice variant (p=3.5E⁻²⁰ and p=1.7E⁻¹⁷, respectively) (FIG. 2A). Levels of the Δ9 splice variant were markedly higher in rs5883T [SEQ ID NO:34] carriers (mean 39%, range 25-48% of total CETP mRNA) and in rs9930761C [SEQ ID NO:35] carriers (mean 36%, range 18-48%; compared to non-carriers (mean 20%, range 10-31%) (FIG. 2B).

There were no samples homozygous for the minor allele. Two subjects were heterozygous for rs9930761 [SEQ ID NO:35] but not rs5883 [SEQ ID NO:34]. Both livers had a relatively low content of the Δ9 splice variant (18%), accounting for the lower p value of rs9930761 [SEQ ID NO:35] than of rs5883 [SEQ ID NO:34].

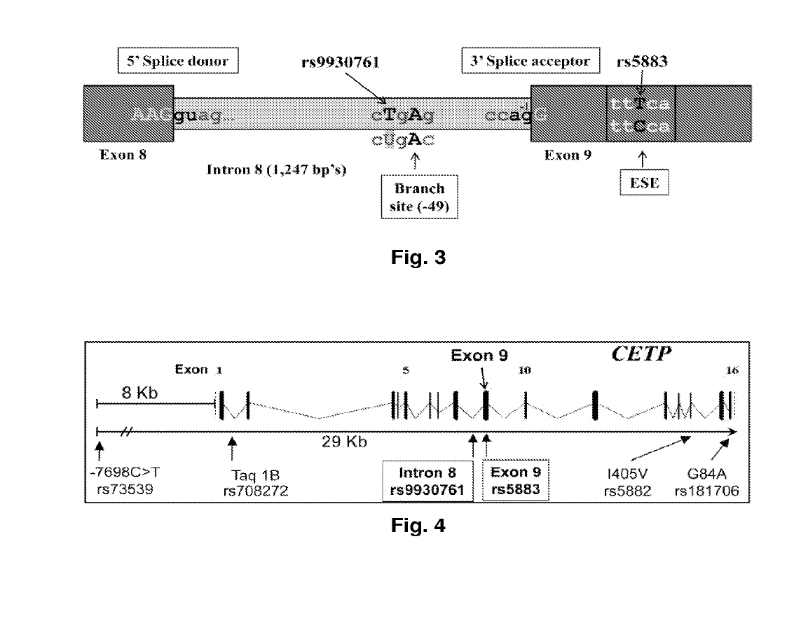

Both rs9930761 [SEQ ID NO:35] and the synonymous SNP rs5883 [SEQ ID NO:34] have impact on the splicing process. The exonic rs5883 [SEQ ID NO:34] minor allele disrupts an exonic splicing enhancer site for SC35 (GTCTTCCA>GTCTTTCA) (ESE finder site (rulai.cshl.edu/cgi-bin/tools/ESE3/esefinder.cgi?process=home) (FIG. 3).

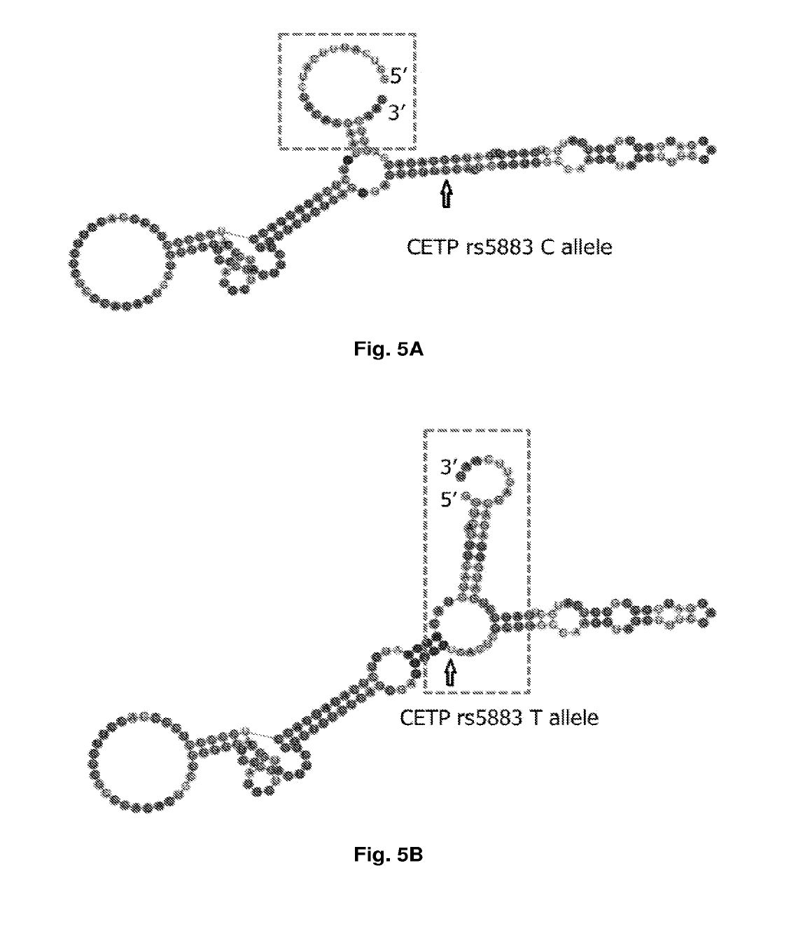

In addition, the rs5883 [SEQ ID NO:34] SNP alters predicted mRNA folding throughout exon 9, as calculated with Mfold (FIGS. 5A-5B). The minor C allele of rs9930761 [SEQ ID NO:35] disrupts a predicted splicing branch point (FIG. 3). A minigene cDNA construct containing exons 8-10 plus intervening introns, when transfected in vitro, also yielded both splice variants; however, even the wild-type sequence already produced mostly Δ9 splice variant (80-90% of total CETP minigene mRNA).

CETP Haplotypes

While pair-wise LD analysis confirms the presence of two main 5′ and 3′ haplotypes blocks, a 6-SNP haplotype analysis (rs173539 [SEQ ID NO:32], rs708272 [SEQ ID NO:38] (Taq1B), rs9930761 [SEQ ID NO:35] (or rs5883 [SEQ ID NO:34]), rs5882 [SEQ ID NO:41] (I1405V), and rs1801706 [SEQ ID NO:43] (G84A)) reveals the presence of only a few long-range haplotypes (FIG. 19—5). The minor C/T alleles of rs9930761 [SEQ ID NO:35]/rs5883 [SEQ ID NO:34] are nearly exclusively embedded in a haplotype consisting of the wild-type alleles of Taq1B (intron 1) and rs173539 [SEQ ID NO:32] (upstream enhancer region), and the minor alleles of I1405V (G) and G84A (A) accounting for weak associations of I1405V and G84A with splicing (FIG. 2A). Since the wild-type alleles of Taq1B and rs173539 [SEQ ID NO:32] (in high LD with rs247616 [SEQ ID NO:31]) are associated with higher CETP levels and reduced HDL-C, the effects of rs5883 [SEQ ID NO:34]/rs9930761 [SEQ ID NO:35] on HDLC must be considered conditional on the upstream promoter SNPs.

Association of rs5883 [SEQ ID NO:34]/rs9930761 [SEQ ID NO:35] with HDL-C levels in Whitehall II.

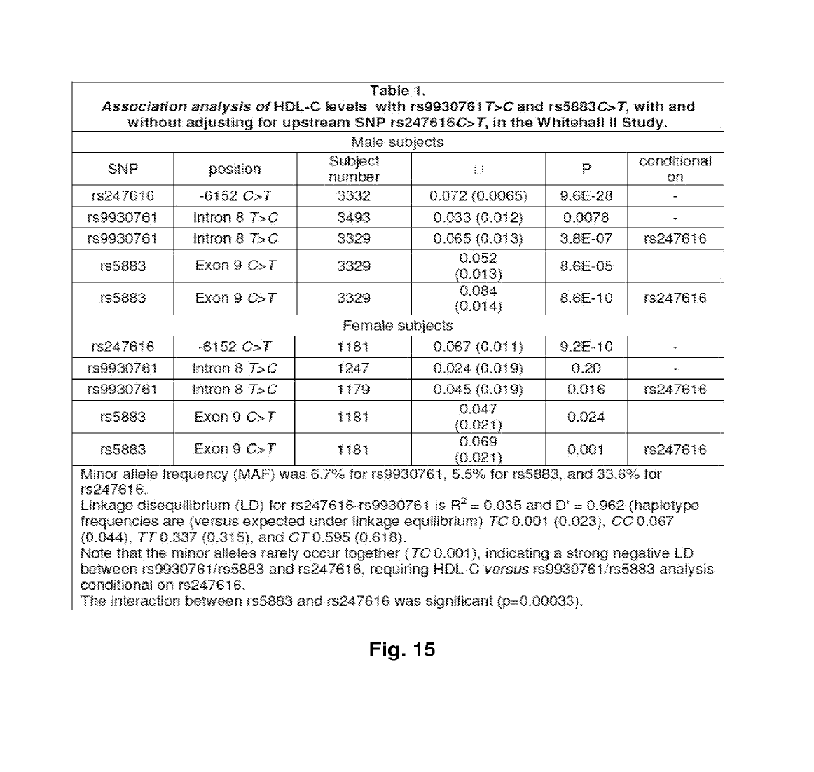

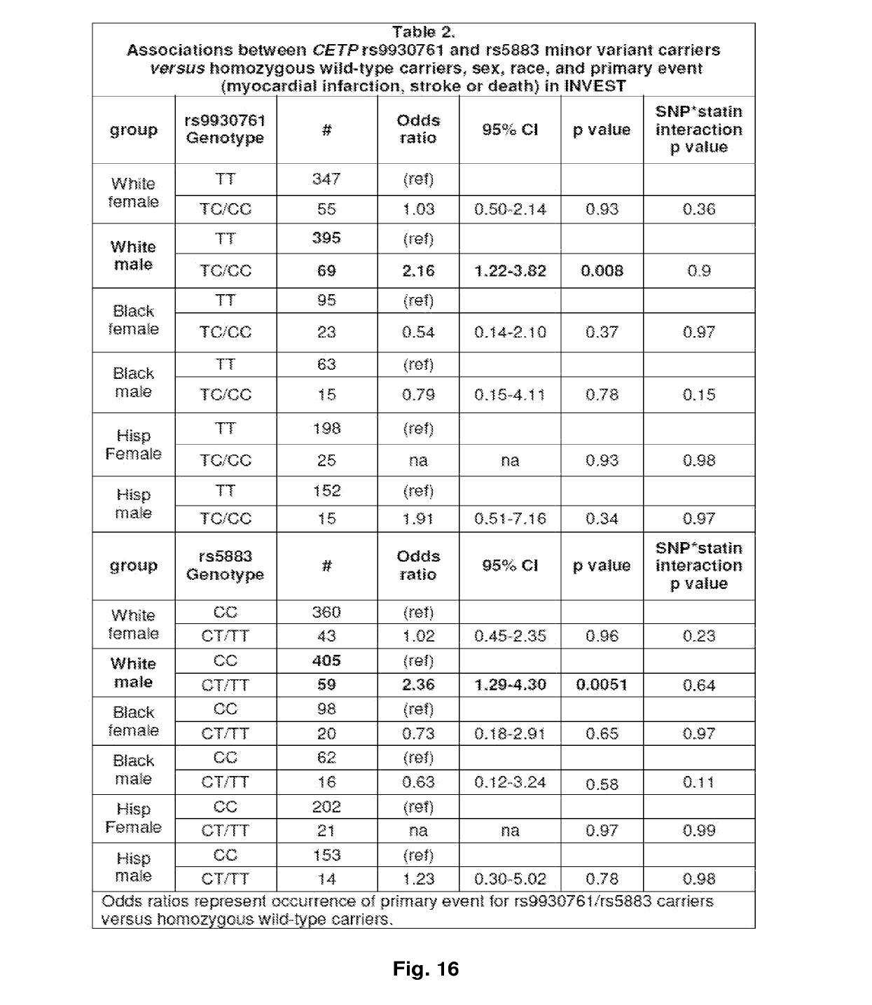

Of 95 CETP SNPs genotyped in 4,745 subjects, many SNPs were strongly associated with HDL-C, largely owing to high LD among them (e.g., rs247616 [SEQ ID NO:31] p=7.18E-29), while, rs5883 [SEQ ID NO:34] and rs9930761 [SEQ ID NO:35] had lower significance (p=6.09×10E-6, and p=0.0012, respectively) (FIG. 20A—Table 6A). The better score for rs5883 [SEQ ID NO:34] (MAF 5.5%) versus rs9930761 [SEQ ID NO:35] (6.7%) in this cohort supports a critical role for rs5883 [SEQ ID NO:34], while a contribution from rs9930761 [SEQ ID NO:35] may also have a role. The lower overall significance for rs5883 [SEQ ID NO:34] and rs9930761 [SEQ ID NO:35] is partially accounted for by low allele frequency compared to enhancer region SNP rs247616 [SEQ ID NO:31] (33.6%). The inventors then determined the associations of rs5883 [SEQ ID NO:34]/rs9930761 [SEQ ID NO:35] with HDL-C by adjusting for rs247616 [SEQ ID NO:31], grouped by sex (females have higher HDL-C levels than males) (FIG. 15—Table 1). The p values for both rs5883 [SEQ ID NO:34] and rs9930761 [SEQ ID NO:35] in males, when made contingent upon rs247616 [SEQ ID NO:31], decreased to p=8.6E-10 and 3.8E-07, respectively (FIG. 15—Table 1).

Each minor allele of either rs5883 [SEQ ID NO:34]/rs9930761 [SEQ ID NO:35] or rs247616 [SEQ ID NO:31] was independently associated with a substantial increase in HDL-C (˜0.1 mmol/L/minor allele) (FIG. 20B—Table 6B), showing significant interactions between them (p=0.00033; FIG. 20C—Table 6C).

Effect of CETP rs5883 [SEQ ID NO:34]/rs9930761 [SEQ ID NO:35]on Risk of Myocardial Infarction (MI) and Other Primary Events in INVEST-GENES

A nested case-control study specifically confirmed that rs5883 [SEQ ID NO:34]/rs9930761 [SEQ ID NO:35] affects primary outcome events (cases: MI, stroke or all-cause mortality) in INVEST-GENES subjects. With stratification by genotype, sex and race, significant associations were observed only in the Caucasian group (866 subjects, FIG. 21A—Table 7A; other groups were too small). White male subjects, but not females, carrying the minor rs5883T [SEQ ID NO:34] and rs9930761C [SEQ ID NO:35] alleles (MAF 6.0% and 7.3%, respectively; D′=1), had significantly increased risk of progression to first event (males p=0.0018-0.0019, respectively, females p=0.73-0.90) (FIG. 16—Table 2, FIG. 21A—Table 7A). The odds ratios for rs5883T [SEQ ID NO:34] and rs9930761C [SEQ ID NO:35] male carriers were 2.36 and 2.24, respectively (95% CI 1.29-4.30 and 1.28-3.91; p=0.0051 and 0.008).

Risk for white males without statin therapy was also substantial (OR 2.0; p=0.034), but risk in the smaller statin-treated male group did not reach significance (rs9930761 [SEQ ID NO:35] carriers (OR 2.8; p=0.089). Therefore, rs5883 [SEQ ID NO:34]/rs9930761 [SEQ ID NO:35] is a general risk factor for male subjects.

The associations of additional CETP SNPs with outcomes are shown in FIG. 21A—Table 7A, separated by males and females. Sex-dependent unadjusted p values of p<0.05 were observed for several SNPs, e.g., enhancer region rs12708967 [SEQ ID NO:50] (males p=0.012 and females p=0.77). The same SNPs also showed highly significant associations HDL levels (rs12708967 [SEQ ID NO:50] p=1.8E-19) (FIG. 21B—Table 7B). However, some enhancer/promoter region SNPs scored only nominally significant in males and others in females (e.g., rs6499861 [SEQ ID NO:51]), with p values that do not survive multiple hypotheses adjustments. Moreover, enhancer region rs247616 [SEQ ID NO:31] (strongly associated with AEI and HDL-C) failed to show significant association in NVEST-GENES (p=0.592 in males and p=0.067 in females). These results show that the promoter/enhancer SNPs did not show a detectable effect on outcomes in INVEST-GENES, in contrast to strong effects on HDL-C.

### Discussion of Example 1

This Example identifies two CETP SNPs strongly associated with splicing to a Δ9 CETP protein thought to act in a dominant-negative fashion. Both rs5883 [SEQ ID NO:34] and rs9930761 [SEQ ID NO:35] show significant associations with HDL and clinical outcomes in cardiovascular risk subjects.

Previously described CETP polymorphisms in a 5′ haplotype block affecting transcription also score highly with respect to HDL levels but failed to carry significant associations with clinical outcomes. Allelic CETP mRNA ratio analysis in human livers identified a region 2.5-7 kb 5′ upstream of the transcription start site, with at least three abundant SNPs, including rs247616 [SEQ ID NO:31], that are strong candidates as regulatory factors.

Identification of Promoter/Enhancer SNPs Affecting CETP mRNA Expression

Using allelic CETP mRNA ratios measured in human livers, the inventors herein have now identified at least three upstream promoter/enhancer SNPs (rs173539 [SEQ ID NO:32], rs247616 [SEQ ID NO:311, and rs3764261 [SEQ ID NO:33]) strongly associated with expression.

rs5883 [SEQ ID NO:34]/rs9930761 [SEQ ID NO:35] Disrupts CETP mRNA Splicing to Yield the Δ9 Splice Variant

Formation of the Δ9 splice variant in human livers was associated with two SNPs in high LD (D′=1) with each other, intron 8 (rs9930761T>C [SEQ ID NO:35]; 5-7% allele frequency in Caucasians and ˜11% in subjects of African descent) and exon 9 (rs5883 [SEQ ID NO:34], with slightly lower minor allele frequency). It was discovered that rs5883 [SEQ ID NO:34] is necessary for enhanced deletion of exon 9, judged by the relatively low Δ9 splice variant content in two livers heterozygous only for rs9930761 [SEQ ID NO:35] but not rs5883 [SEQ ID NO:34]. However, all livers with high Δ9 splice variant content were heterozygous for both rs9930761 [SEQ ID NO:35] and rs5883 [SEQ ID NO:34], showing that both are involved in achieving effective skipping of exon 9. It is remarkable that the LD between the two SNPs is nearly complete even in African populations, residing predominantly in a single haplotype stretching over at least 20 kb, showing this represents an evolutionarily conserved haplotype.

The rs5883T [SEQ ID NO:34] allele disrupts an ESE enhancer consensus site and alters RNA folding of the entire exon 9 (FIG. 5). The rs9930761C [SEQ ID NO:35] allele, located 40 bp's upstream of exon 9, modulates a splicing branch point consensus sequence CT>CRAY required in mammalian splicing (FIG. 3). With the intron 8 wild-type sequence CTGAG already predicted to be a weak branch point, low level of exon 9 skipping does occur in livers. Moreover, transfection of a minigene construct resulted in predominant exon 9 skipping (80-90%; data not shown), showing that the splice branch point is already compromised in the wild-type sequence. As none of the livers were homozygous for the minor splicing allele, the maximum measured level of 48% Δ9 formation in heterozygotes represents a high degree of exon 9 skipping of the variant rs5883 [SEQ ID NO:34]/rs9930761 [SEQ ID NO:35] alleles. No other CETP SNPs account for the observed genetic effect on splicing. While not wishing to be bound by theory, the inventor wherein believe that the biological effect of exon 9 deletion may be amplified by dominant-negative interactions through heterodimer formation of the Δ9 splice variant with full-length CETP, preventing cellular exit of mature CETP protein.

Association of Promoter/Enhancer SNPs and rs5883 [SEQ ID NO:34]/rs9930761 [SEQ ID NO:35] with HDL-C Levels.

Strong HDL-C associations were observed with a series of promoter/enhancer SNPs present at high frequency (>30%) (e.g., for rs247616 [SEQ ID NO:31] p=6.14E-29), whereas the association was relatively weaker for rs9930761 [SEQ ID NO:35] and rs5883 [SEQ ID NO:34] (FIG. 20A—Table 6A), indicating less clinical relevance. However, haplotype estimates revealed that rs5883T [SEQ ID NO:34]/rs9930761C [SEQ ID NO:35] predominantly share a haplotype consisting of the main wild-type alleles (associated with high HDL-levels) of all high scoring SNPs in the promoter/enhancer region (FIG. 19—Table 5). Adjusting for enhancer SNP rs247616 [SEQ ID NO:31], the HDL-C association strengthened for both rs9930761 [SEQ ID NO:35] and rs5883 [SEQ ID NO:34] (p=8.6E-10 in males) (FIG. 15—Table 1).

rs5883 [SEQ ID NO:34] consistently scored with greater significance than rs9930761 [SEQ ID NO:35], the latter with ˜1% greater allele frequency, showing that rs5883 [SEQ ID NO:34] is necessary for exon 9 skipping, while rs9930761 [SEQ ID NO:35] is insufficient but may also be involved or required. A strong interaction was observed for effects on HDL-C between rs247616 [SEQ ID NO:31] and the splicing SNPs (interaction model p=0.00033), consistent with their location on different haplotypes and mechanistically distinct effects.

rs9930761 [SEQ ID NO:35] and rs5883 [SEQ ID NO:34] allele frequencies differ between ethnic groups, while maintaining high LD and r², ranging from 0% (Asians) and 7.5% in Caucasians to 12.5% (Yoruban). In a Yoruban population, rs9930761 [SEQ ID NO:35] allele frequency was reported to be 4% in subjects with low HDL levels, and 16% in those with high HDL, showing a large effect on HDL in this population (FIG. 22—Table 8).

Thus, rs5883 [SEQ ID NO:34] and rs9930761 [SEQ ID NO:35] have strong effects on HDL-C, independent of the upstream promoter/enhancer SNPs (for which Taq1B has served as a surrogate if not suboptimal marker).

CETP Genotype Effect on Progression to Event in the INVEST-GENE Study

While the genotyping array contains 95 CETP SNPs, the present study on subjects with pre-existing coronary artery disease and high blood pressure focuses on whether the newly discovered splicing SNPs have clinical relevance. Even though present at relatively low allele frequency, rs5883T [SEQ ID NO:34]/rs9930761C [SEQ ID NO:35] were significantly associated with risk for an event (such as MI, stroke, or death), in males (rs5883 [SEQ ID NO:34] in Caucasians, OR 2.36; 95% CI 1.29-4.3, p=0.0051) (FIG. 16—Table 2 and FIG. 21A—Table 7A). As no significant association was observed in females, the inventors herein now believe that this effect is sex-dependent.

This example shows that rs5883 [SEQ ID NO:34]/rs9930761 [SEQ ID NO:35] are predictive of increased primary events (MI, stroke and death) in male at-risk subjects. The results reported here support CETP variants as a potential disease markers and predictor of statin therapy outcome, and in evaluating CETP inhibitor drugs, such as torcetrapib, in the treatment of coronary artery disease.

Materials and Methods

Human Liver Tissues

Frozen human liver samples (125 normal liver biopsy and autopsy samples) were from The Cooperative Human Tissue Network, Midwestern and Western Divisions, using IRB approved protocols. Collection to processing intervals were <24 hours.

Whitehall II Study

Between 1985 and 1988, all civil servants aged between 35 and 55 years in 20 departments in London were invited to a medical examination at their workplace. Follow-up visits took place every two years. In the present analysis, CETP association with HDL was limited to white subjects (n=4745).

INVEST-GENES

The International Verapamil SR Trandolapil Study (INVEST) evaluated adverse cardiovascular outcomes following randomized treatment with either an atenolol- or a verapamil-based treatment strategy in 22,576 subjects aged 50 years or older, with documented CAD and essential hypertension as defined by JNC VI. Primary outcomes were first occurrence of all-cause mortality, nonfatal myocardial infarction (MI), or nonfatal stroke. From 5,979 INVEST subjects from 213 sites in the USA and Puerto Rico providing DNA samples under written informed consent for genetic studies, a nested case-control study was designed with 292 INVEST-GENES subjects experiencing primary outcome events during follow-up (cases) and 1168 subjects who did not, frequency-matched to cases for age (by decades), sex, and race/ethnicity in a ratio of approximately 4:1 (controls/cases), an approach shown to yield equivalent results to analyses of the entire cohort.

RNA and DNA Preparation from Liver Tissues

RNA was extracted from 125 biopsy or autopsy liver tissues. Frozen tissue samples were pulverized under liquid nitrogen. RNA was extracted using TRIZOL™, followed by Dnase treatment and Qiagen Rneasy column purification. cDNA was generated from 1 μg purified mRNA using the Superscript II kit (Invitrogen, Carlsbad, Calif.) with oligo-dT and CETP gene-specific primers. Liver DNA was prepared by digestion of pulverized frozen liver tissue in Tris EDTA buffer containing proteinase K and SDS, followed by NaCl salting-out of proteins and ethanol precipitation.

Quantitative RT-PCR (qRT-PCR) Analysis of CETP mRNA

Real-time PCR was performed on an ABI 7000 instrument using ABI SYBR Green master mix (primer sequences in FIG. 17—Table 3). Beta-actin and CETP-specific primers amplified with >99% efficiency.

Allelic CETP mRNA Expression in Human Liver Tissues

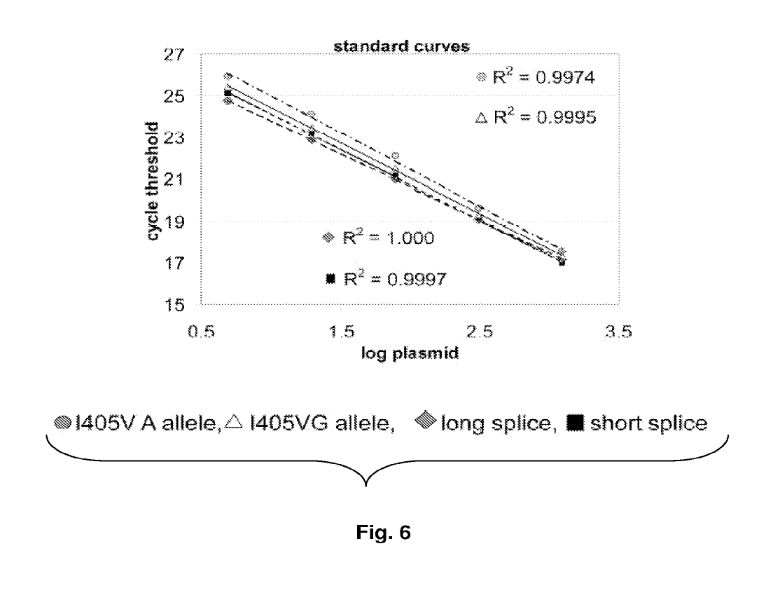

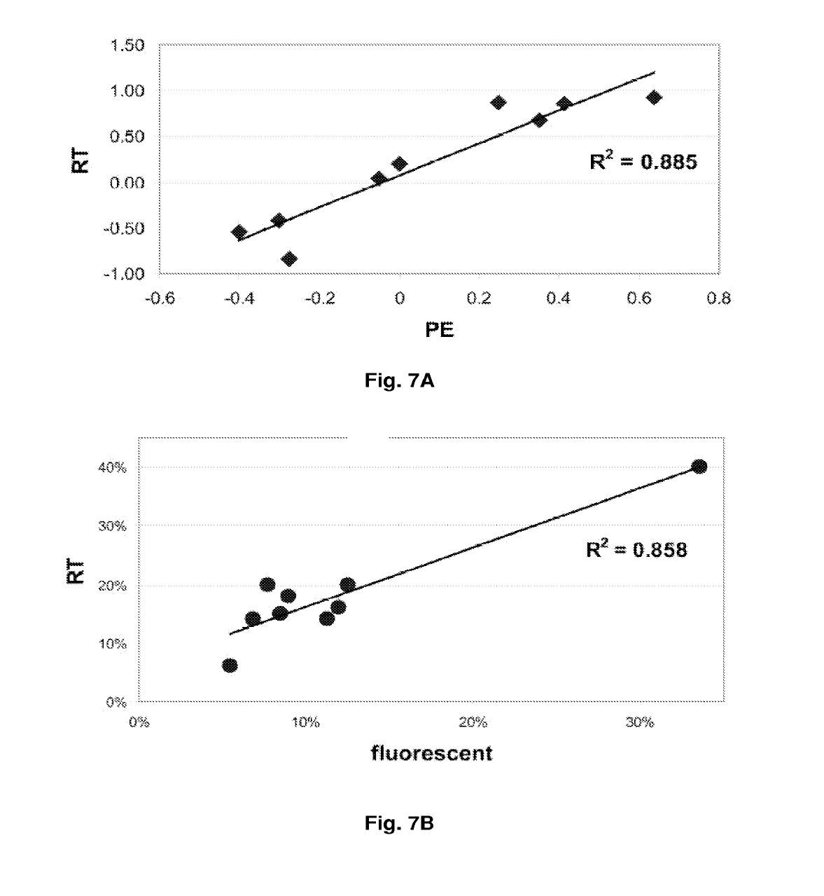

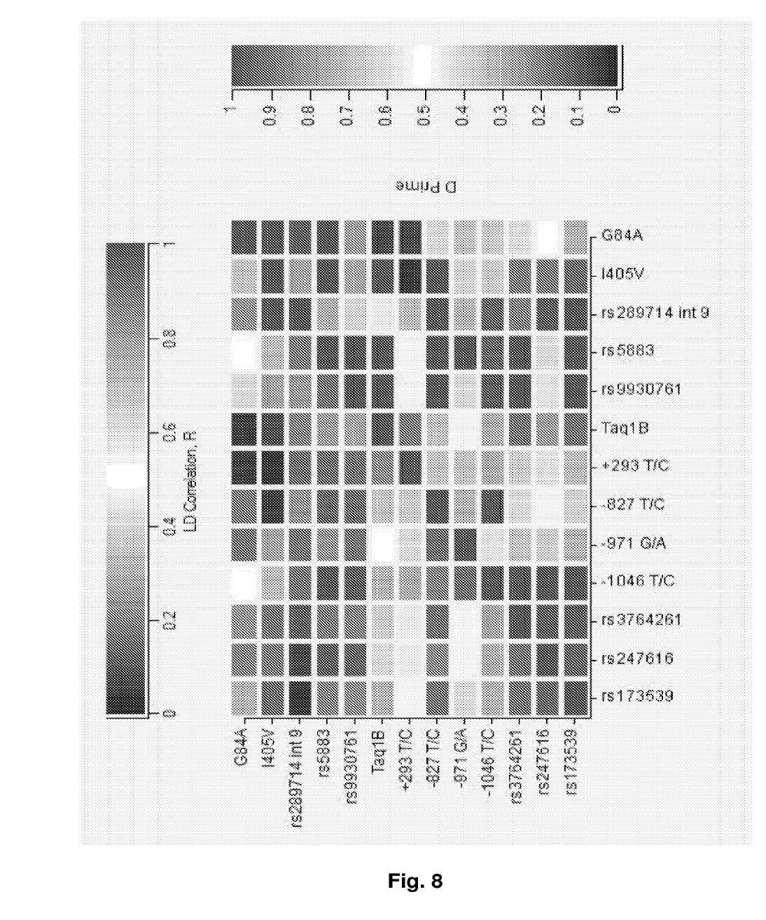

As an accurate measure of cis-acting regulatory factors, allelic mRNA ratios were measured after conversion to cDNAs and PCR amplification, using a primer extension method (SNaPshot, Life Technologies, Foster City, Calif.). Allelic mRNA ratios were normalized to gDNA ratios (standardized to 1, SD±0.03). Standard curves with cloned cDNAs representing the two alleles gave straight lines with R²=0.99 (FIG. 6). Standard deviations for each subject allelic mRNA ratio ranged from 3-8%. The inventors also employed allele-selective qRT-PCR, which yielded similar allelic mRNA ratios compared to SNaPshot (R=0.89, FIGS. 7A-7B), supporting accuracy of the results. FIG. 8 shows an LD structure map showing r² and D′ values between all 13 SNPs.

Quantitative Analysis of CETP Δ9 Splice Variant Using RT-PCR with Fluorescently Labeled Primers and Splice Variant-Specific qRT-PCR

Splice variants were simultaneously PCR amplified using one set of splice-specific (Δ9 and long splice variant) forward primers and a common 6-FAM-fluorescently labeled reverse primer in a Sigma Ready Mix solution (Sigma Aldrich, St. Louis, Mo.) primers: FIG. 17—Table 3). Liver cDNA was PCR-amplified for 25 cycles yielding amplicons of 409 bp (Δ9) and 450 bp (long), and the fluorescent peaks were analyzed on an ABI 3730 sequencer, using Gene Mapper version 3.1 (Life Technologies, Foster City, Calif.). Independent analysis using quantitative RT-PCR with SYBR-Green gave similar results (R²=0.85; FIGS. 7A-7B). Cloned fragments of full length CETP and the Δ9 splice variant were used to establish linearity of the assay (R²>0.99) FIG. 6).

Genotyping

Multiple methods, e.g., allele-specific PCR, were used to genotype 13 CETP SNPs in liver (see FIG. 18—Table 4). All clinical study cohorts were genotyped using the Illumina (Illumina Inc. San Diego, Calif., USA) IBC Candidate Gene array, version 2 (WHII) or version 3 (INVEST-GENES), representing between 49,094 (v2) to 53,831 (v3) SNPs covering ˜2,100 cardiovascular candidate loci, with 95 CETP SNPs. SNPs with a less than 95% call rate were excluded. Subjects with a call rate less than 95%, related samples, and population outliers were excluded using PLINK and EIGENSTRAT. Hardy-Weinberg Equilibrium was evaluated using chi-squared test.

Sequencing CETP Exon 8 to Exon 10 Splice Region

The inventors sequenced a 3.1 kilobase fragment of the CETP exon 8-10 region in 6 livers with high or low Δ9 splice formation. Three segments of approximately 1200 bases each were PCR amplified and Sanger sequenced in both directions on an ABI 3730.

Statistical Methods

Statistical analysis of associations between CETP polymorphisms and allelic mRNA ratios or percent splice Δ9 splice variant was performed using Helix Tree software package (Golden Helix, Inc., Bozeman, Mont.). Pair-wise linkage disequilibrium (LD) was determined for each combination of liver SNPs, also using Helix Tree software. Haplotypes were predicted with the Helix Tree estimation-maximization algorithm.

Association Between CETP SNPs and HDL-C in the Whitehall II Study

Two (rs173539 [SEQ ID NO:32] and rs3816117 [SEQ ID NO:52]) out of 13 SNPs investigated in vitro were not present on the Illumina IBC Candidate Gene array, version 2. These two, and additional CETP SNPs, were imputed from the HapMap3 and 1000 Genomes Project CEU datasets using the IMPUTEv2 software (mathgen.stats.ox.ac.uk/impute/impute_v2.html). CETP SNP association analysis with log-transformed HDL was carried out using PLINK, assuming an additive model. Analysis was performed in men and women separately with no adjustment for any covariates. A further analysis was carried out conditional on the enhancer region SNP rs247616 [SEQ ID NO:31], which itself was strongly associated with HDL levels.

INVEST-GENES

Baseline characteristics were compared using chi-squared test or analysis of variance. To minimize population stratification in the diverse population of INVEST, all analyses were conducted separately by race/ethnicity. For the INVEST-GENES case-control samples, adjusted odds ratios (ORs) and 95% confidence intervals (CIs) for occurrence of the primary outcome were calculated using logistic regression.

Processes for SNP Selection

Group 1 (n=435 loci); genes and regions with a high likelihood of functional significance, including established mediators of vascular disease, loci derived from GWAS and those shown to be associated with phenotypes of interest. Tag SNPs for these loci were selected to capture known variation with MAF>0.02 and an r2 of at least 0.8 in HapMap populations and SeattleSNPs where available.

Group 2 (n=1,349 loci); candidate loci that are potentially involved in phenotypes of interest or established loci that required very large numbers of tagging SNPs. SNPs for these loci were selected for MAFs>0.05 with an r2 of at least 0.5 in HapMap populations and SeattleSNPs where available.

Group 3 (n=232 loci); comprised mainly of the larger genes (>100 kb) which were of lower interest a priori to the consortium investigators. Only non-synonymous SNPs (nsSNPs) and known functional variants of MAF>0.01 were captured for these loci.

Assays for specific SNPs of known or putative functionality and those shown to be highly associated with vascular disease from literature searching were directly ‘forced’ into the array content, with the aim of facilitating more powerful downstream meta-analyses with previously published data. nsSNPs and known functional variants of MAF>0.01 were selected where possible for all genes of interest.

SNPs from Group 1 and 2 loci were first chosen using the TAGGER software. Assays for SNPs in Group 1 loci were designed to be inclusive of the intronic, exonic, untranslated regions (UTRs) and 5 kb of the proximal promoter regions derived from NCBI build 35 with intronic, exonic and flanking UTRs covered for the ‘Group 2’ loci. This approach generated a set of tag SNPs and multimarker predictors that capture variation in the four HapMap populations (CEU, Centre d'Etude du Polymorphisme Humain collection; CHB, Han Chinese in Beijing, China; JPT, Japanese subjects from Tokyo, YRI, Yoruba from Ibadan, Nigeria; HapMap Data release 21/phase II July 2006 on NCBI build 35, dbSNP build 125). Where available, we also employed SeattleSNPs (pga.gs.washington.edu) and Environmental Genome Project (EGP), (egp.gs.washington.edu) resequencing data to identify additional tags, not represented in the HapMap populations, using ldSelect. The inventors choose SNPs that were observed at least twice in unrelated subjects.

SNP selection information taken from: Keating B J, Tischfield S, Murray S S, BhangaleT, Price T S, et al. (2008) Concept, Design and Implementation of a Cardiovascular Gene-Centric 50 K SNP Array for Large-Scale Genomic Association Studies. PLoS ONE 3(10): e3583. doi:10.1371/journal.pone.0003583.

### Example 2

In a registry cohort of ˜1414 documented CAD patients and 478 healthy controls with no CAD, SNPs rs247616 [SEQ ID NO:31] and rs9930761 [SEQ ID NO:35] were tested for affecting risk of CAD.

Male homozygous carriers of rs247616 [SEQ ID NO:31] had significantly lower CAD risk (OR=0.56, CI 0.36-0.88, p=0.013), an effect not observed in females in this cohort. Also, in this cohort, rs9930761 [SEQ ID NO:35] (less frequent) also did not score significantly. Since the effect of the minor alleles of rs247616 [SEQ ID NO:31] and rs9930761 [SEQ ID NO:35] on cholesterol levels is similar, the inventors also tested the additive effects of the minor alleles in both SNPs.

Male subjects with 2 or more minor alleles from either SNP also appeared to have reduced CAD risk (OR=0.62, CI 0.42-0.90, p=0.013). Therefore, these SNPs are now believed by the inventors herein to predict CAD risk in a healthy population.

### Example 3

In a Dutch registry study, ˜1632 subjects with elevated cholesterol levels (at increased risk of CAD), 632 were treated with statins and 1,000 were not (controls) were studied. Statin treatment was highly effective in preventing cardiovascular events (myocardial infarction) (OR=0.35 (CI 0.28-0.45, p=0.00) across all subjects.

However, male carriers homozygous for the minor allele of rs3764261 [SEQ ID NO:33] (in high LD with rs247616 [SEQ ID NO:31] with r²=0.95) did not benefit from statin therapy, showing no reduction in cardiovascular events (OR=09 (CI 0.51-2.33, p=0.33), a finding in this cohort that applied only to male subjects but not to females.

### Example 4

Assays, Sequences and Primers

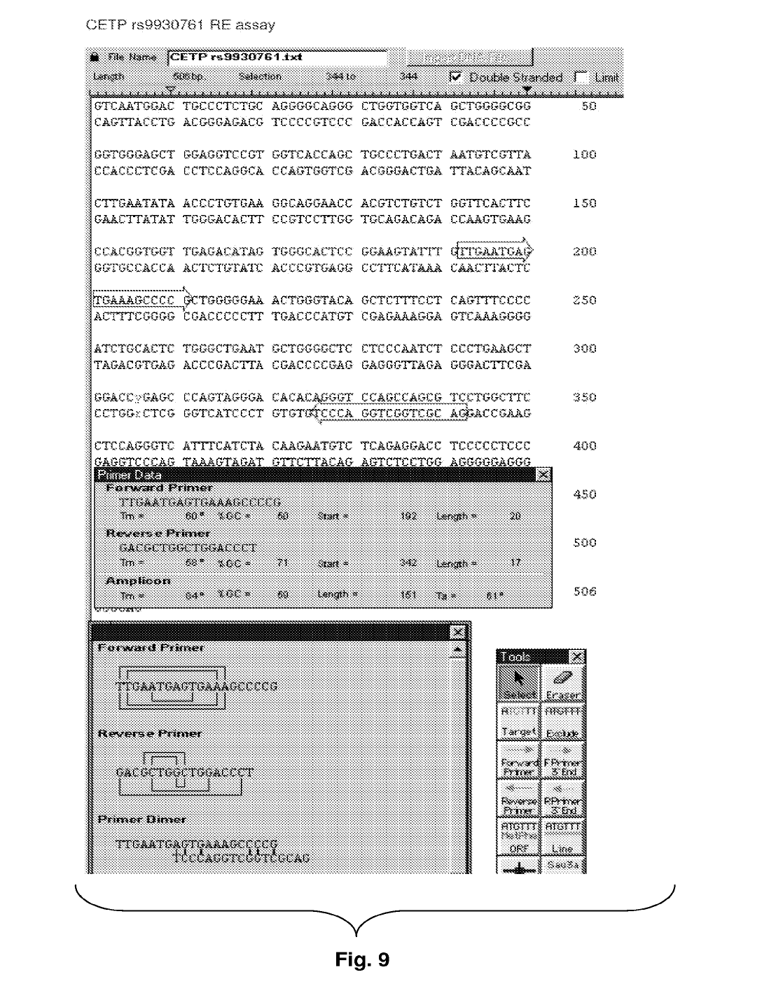

FIG. 9 shows the CETP rs9930761 [SEQ ID NO:35] RE assay FIG. 9 discloses the “CETP” rs9930761 RE assay sequence as nucleotides 1-400 of SEQ ID NO: 12, as well as the forward primer, reverse primer and primer dimer sequences as SEQ ID NOs: 10, 11, 10, 11, 10 and 11, respectively, in order of appearance.

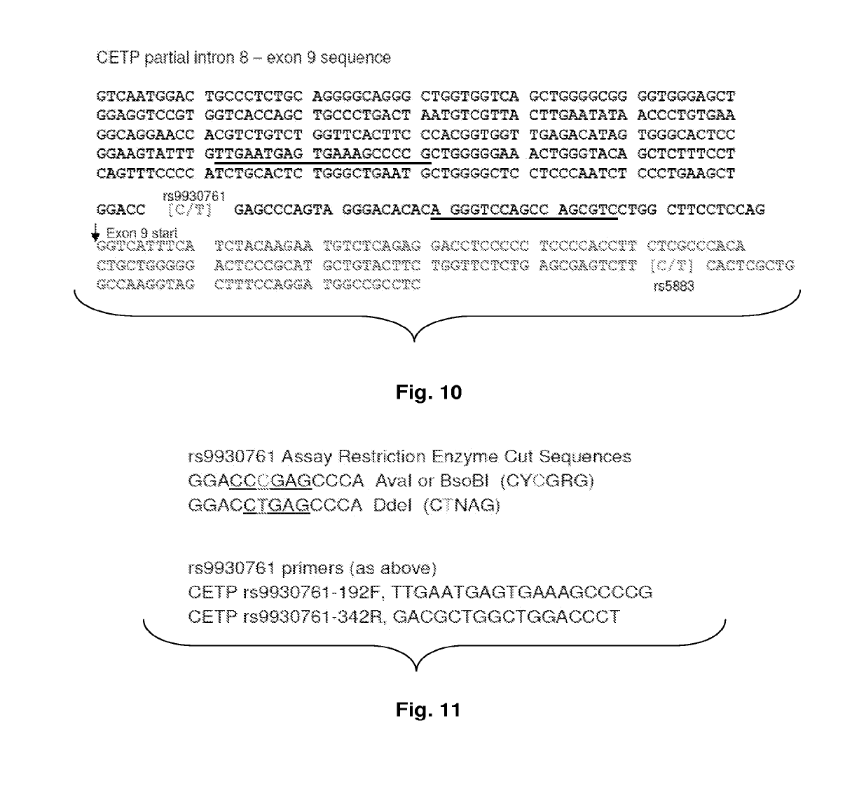

FIG. 10 shows the CETP partial intron 8-exon 9 sequence [SEQ ID NO:12].

FIG. 11 shows the rs9930761 [SEQ ID NO:35] assay restriction cut enzyme sequences [SEQ ID NOs:13-14] and rs9930761 [SEQ ID NO:35] primers [SEQ ID NOs:10-11].

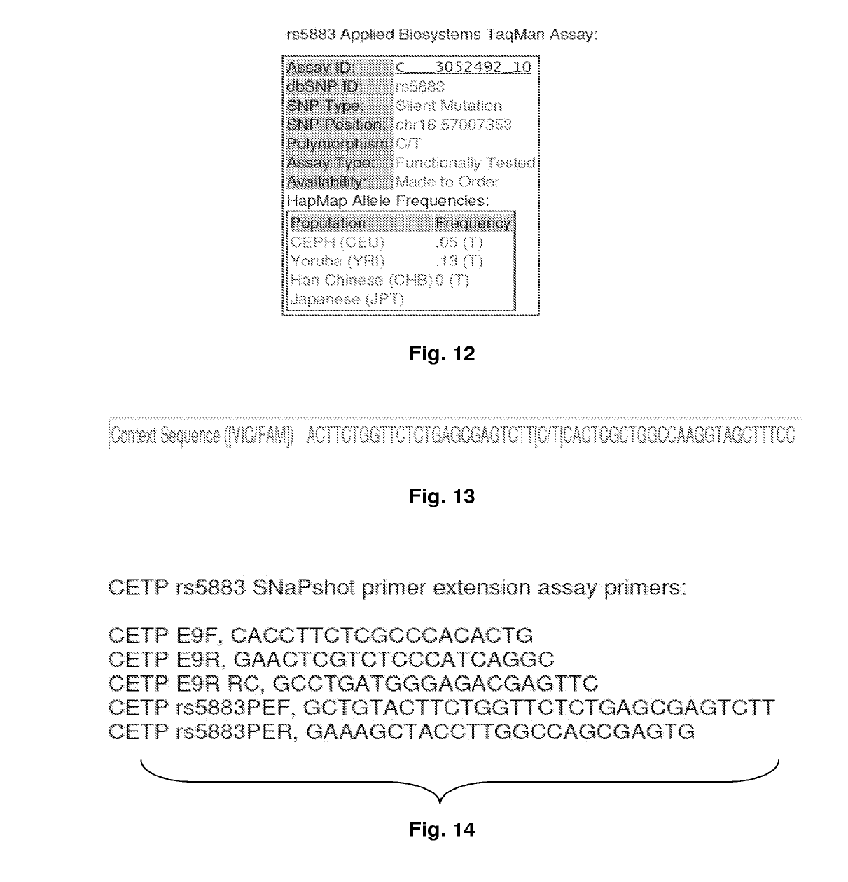

FIG. 12 shows the rs5883 [SEQ ID NO:34] Applied Biosystems TaqMan Assay.

FIG. 13 shows the Context Sequence ([VIC/FAM]) [SEQ ID NO:15].

FIG. 14 shows the CETP rs5883 [SEQ ID NO:34] SnaPshot primer extension assay primers [SEQ ID NOs:16-20].

Thus there is also provided herein markers and methods for the diagnosis of a coronary artery disease (CAD) or a predisposition therefor in a mammal, particularly in a human being, comprising determining the presence of at least one polymorphism of CETP. In a particular embodiment, the at least one polymorphism is located within an intron of CETP.

Also provided are markers for, and methods of, distinguishing subjects having an increased susceptibility to CAD using a DNA microarray for detecting one or more coronary artery disease (CAD)-associated polymorphisms in a genetic sample, comprising the steps of: providing a nucleic acid sample; performing a hybridization to form a double-stranded nucleic acid between the nucleic acid sample and a probe; and detecting the hybridization. In certain embodiments, the hybridization is detected by radioactively, fluorescence, electrically, and/or under stringent conditions.

Also provided is a DNA microarray for detecting one or more coronary artery disease (CAD)-associated polymorphisms in a genetic sample, the DNA microarray being comprised of in situ synthesized oligonucleotides. The DNA microarray can be a randomly or non-randomly assembled bead-based array. The DNA microarray can be comprised of mechanically assembled arrays of spotted material, the spotted material selected from the group of an oligonucleotide, a cDNA clone, and a Polymerase Chain Reaction (PCR) amplicon.

In certain embodiments, the nucleic acid is a nucleic acid extract from a biological sample from the human. Also, in certain embodiments, a combination of polymorphisms in at least one chromosomal copy of the CETP gene is determined.

In certain embodiments, the polymorphism is determined by a genotyping analysis, or by sequencing of the genomic region, or whole genome sequencing.

In certain embodiments, the genotyping analysis comprises an array analysis. In certain embodiments, the genotyping analysis comprises the use of a restriction enzyme assay. In certain embodiments, the genotyping analysis comprises an allele-specific polymerase chain reaction analysis.

In certain embodiments, the allele-specific method is allele-specific probe hybridization, allele-specific primer extension, or allele-specific amplification.

In certain embodiments, the genetic material comprises DNA and/or RNA, and, in certain embodiments, the genetic material is amplified.

### Example 5

Methods for Analyzing

It will be appreciated that the methods described herein can be performed in conjunction with an analysis of other non-genetic factors known to be associated with a subject's likely response to an agent. Such factors include epidemiological risk factors associated with poor response or resistance to an agent.

Such risk factors include, but are not limited to smoking and/or exposure to tobacco smoke, age, sex and familial history. These risk factors can be used to augment an analysis of one or more polymorphisms as herein described when assessing a subject's response to an agent.

As described herein, the effect of more than one responsive or restrictive polymorphisms can be combined to more accurately predict or determine a subject's response to an agent, or their suitability to a treatment regime.

In certain embodiments, the presence of any of the SNPs is correlated with an increased risk for developing cardiac disorders or statin resistance in males subjects already diagnosed with coronary artery disease.

Also described herein are methods for predicting or determining a subject's response to an agent, and to methods for determining a subject's suitability to a treatment regime or intervention. The methods comprise the analysis of polymorphism/s herein shown to be associated with responsiveness to an agent, or the analysis of results obtained from such an analysis. The use of polymorphisms herein shown to be associated with responsiveness to an agent in the assessment of a subject's suitability to a treatment regime or intervention are also provided, as are nucleotide probes and primers, kits, and microarrays suitable for such assessment. Methods of treating subjects having the polymorphisms herein described are also provided. Methods for screening for compounds able to modulate the expression of genes associated with the polymorphisms herein described are also provided.

In addition to identifying responsive or unresponsive subjects, it is possible to segment a population to define a subgroup of the population that is suitable to undergo an intervention. Such an intervention may be a diagnostic intervention, such as imaging test, other screening or diagnostic test (e.g., biochemical or RNA based test), or may be a therapeutic intervention, such as a chemopreventive or chemotherapeutic therapy, or a preventive lifestyle modification (such as stopping smoking or increasing exercise).

In defining such a clinical threshold, people can be prioritized to a particular intervention in such a way to minimize costs or minimize risks of that intervention (for example, the costs of image-based screening or expensive preventive treatment or risk from drug side-effects or risk from radiation exposure).

In determining this threshold, one might aim to maximize the ability of the test to detect the majority of cases (maximize sensitivity) but also to minimize the number of people at low risk that require, or may be are otherwise eligible for, the intervention of interest.

Accordingly, there is also provided herein a method of assessing a subject's suitability for an intervention diagnostic of or therapeutic for a disease or condition associated with platelet aggregation, the method comprising:

a) providing the result of one or more genetic tests of a sample from the subject, and

b) analyzing the result for the presence or absence of one or more responsive polymorphisms or for the presence or absence of one or more resistance polymorphisms, or one or more polymorphisms which are in linkage disequilibrium with any one or more of the polymorphisms;

wherein the presence of one or more responsive polymorphisms is indicative of the subject's suitability for the intervention, and wherein the absence of one or more responsive polymorphisms or the presence of one or more resistance polymorphisms is indicative of the subject's unsuitability for the intervention.

The intervention may be a diagnostic test for the disease, such as a blood test or a CT scan for CAD. Alternatively, the intervention may be a therapy for the disease, such as chemotherapy or radiotherapy, including a preventative therapy for the disease, such as the provision of motivation to the subject to stop smoking and/or start exercising.

In certain embodiments, a method for analyzing includes the steps of:

a) analyzing a sample from a subject with a SNP detection assay to determine that the subject is homozygous for at least one polymorphism in the CETP gene, thereby generating a homozygous genetic analysis result;

b) inputting the homozygous genetic analysis result into a system, wherein the system comprises:

i) a computer processor for receiving, processing, and communicating data,

ii) a storage component for storing data which contains a reference genetic database of results of at least one genetic analysis of homozygosity with respect to response to an agent, and

iii) a computer program, embedded within the computer processor, which is configured to process the homozygous genetic analysis result in the context of the reference database to determine, as an outcome, that the subject will be responsive to the agent;

c) processing the homozygous genetic analysis result with the computer program in the context of the reference database to determine, as an outcome, that the subject is responsive to the agent;

d) communicating the outcome from the computer program; and

e) modulating therapy being administering to the subject.

The implementation of the methods in computer systems and programs as described herein, the data produced by such methods, and the use of such data in the prediction or determination of a subject's response to an agent, or in the determination of a subject's suitability or unsuitability for an intervention diagnostic or therapeutic of a disease or condition associated with CAD are also contemplated.

As used herein, the phrase “assessing a subject's suitability for an intervention” or grammatical equivalents thereof means one or more determinations of whether a given subject is or should be a candidate for an intervention or is not or should not be a candidate for an intervention.

As used herein the term “intervention” includes medical tests, analyses, and treatments, including diagnostic, therapeutic and preventative treatments, and psychological or psychiatric tests, analyses and treatments, including counseling and the like.

### Example 6

Computer-Related Embodiments

It will also be appreciated that the methods described herein are amenable to use with and the results analyzed by computer systems, software and processes. Computer systems, software and processes to identify and analyze genetic polymorphisms are well known in the art. Similarly, implementation of the algorithm utilized to generate a SNP score as described herein in computer systems, software and processes is also contemplated. For example, the results of one or more genetic analyses as described herein may be analyzed using a computer system and processed by such a system utilizing a computer-executable example of the analyses described herein.

Both the SNPs and the results of an analysis of the SNPs may be “provided” in a variety of mediums to facilitate use thereof. As used in this section, “provided” refers to a manufacture, other than an isolated nucleic acid molecule, that contains SNP information. Such a manufacture provides the SNP information in a form that allows a skilled artisan to examine the manufacture using means not directly applicable to examining the SNPs or a subset thereof as they exist in nature or in purified form. The SNP information that may be provided in such a form includes any of the SNP information provided herein such as, for example, polymorphic nucleic acid and/or amino acid sequence information, information about observed SNP alleles, alternative codons, populations, allele frequencies, SNP types, and/or affected proteins, identification as a responsive SNP or a resistance SNP, weightings (for example for use in an combined analysis as described herein), or any other information provided.

In one application of this embodiment, the SNPs and the results of an analysis of the SNPs utilized can be recorded on a computer readable medium. As used herein, “computer readable medium” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon SNP information.

As used herein, “recorded” refers to a process for storing information on computer readable medium. A skilled artisan can readily adopt any of the presently known methods for recording information on computer readable medium to generate manufactures comprising the SNP information.

A variety of data storage structures are available to a skilled artisan for creating a computer readable medium having recorded thereon SNP information. The choice of the data storage structure will generally be based on the means chosen to access the stored information. In addition, a variety of data processor programs and formats can be used to store the SNP information on computer readable medium. For example, sequence information can be represented in a word processing text file, formatted in commercially-available software such as WordPerfect and Microsoft Word, represented in the form of an ASCII file, or stored in a database application, such as OB2, Sybase, Oracle, or the like. A skilled artisan can readily adapt any number of data processor structuring formats (e.g., text file or database) in order to obtain computer readable medium having recorded thereon the SNP information.

By providing the SNPs and/or the results of an analysis of the SNPs utilized in computer readable form, a skilled artisan can routinely access the SNP information for a variety of purposes. Computer software is publicly available which allows a skilled artisan to access sequence information provided in a computer readable medium. Examples of publicly available computer software include BLAST (Altschul et at, J. Mol. Biol. 215:403-410 (1990)) and BLAZE (Brutlag et at, Comp. Chem. 17:203-207 (1993)) search algorithms.

Also provided herein are systems, particularly computer-based systems, which contain the SNP information described herein. Such systems may be designed to store and/or analyze information on, for example, a number of SNP positions, or information on SNP genotypes from a number of subjects. The SNP information represents a valuable information source. The SNP information stored/analyzed in a computer-based system may be used for such applications as predicting a subject's likely responsiveness to an agent, in addition to computer-intensive applications as determining or analyzing SNP allele frequencies in a population, mapping disease genes, genotype-phenotype association studies, grouping SNPs into haplotypes, correlating SNP haplotypes with response to particular drugs, or for various other bioinformatic, pharmacogenomic, drug development, or human identification/forensic applications.

As used herein, “a computer-based system” refers to the hardware, software, and data storage used to analyze the SNP information. The minimum hardware of the computer-based systems can include a central processing unit (CPU), an input, an output, and data storage. A skilled artisan can readily appreciate that any one of the currently available computer-based systems are suitable for use. Such a system can be changed into a system by utilizing the SNP information without any experimentation.

As used herein, “data storage” refers to memory which can store SNP information, or a memory access facility which can access manufactures having recorded thereon the SNP information.

The one or more programs or algorithms are implemented on the computer-based system to identify or analyze the SNP information stored within the data storage. For example, such programs or algorithms can be used to determine which nucleotide is present at a particular SNP position in a target sequence, to analyze the results of a genetic analysis of the SNPs described herein, or to derive a SNP score as described herein. As used herein, a “target sequence” can be any DNA sequence containing the SNP position(s) to be analyzed, searched or queried.

A variety of structural formats for the input and output can be used to input and output the information in the computer-based systems. An exemplary format for an output is a display that depicts the SNP information, such as the presence or absence of specified nucleotides (alleles) at particular SNP positions of interest. Such presentation can provide a rapid, binary scoring system for many SNPs or subjects simultaneously. It will be appreciated that such output may be accessed remotely, for example over a LAN or the internet. For example, given the nature of SNP information, such remote accessing of such output or of the computer system itself is available only to verified users so that the security of the SNP information and/or the computer system is maintained. Methods to control access to computer systems and the data residing thereon arc well-known in the art, and arc amenable to the embodiments described herein.

Accordingly, there is provided herein a system for determining a subject's response to an agent, the system comprising: computer processor means for receiving, processing and communicating data; storage means for storing data including a reference genetic database of the results of at least one genetic analysis with respect to response to an agent or with respect to a disease or condition associated with CAD, and optionally a reference non-genetic database of non-genetic risk factors for a disease or condition associated with CAD; and, a computer program embedded within the computer processor which, once data consisting of or including the result of a genetic analysis for which data is included in the reference genetic database is received, processes the data in the context of the reference databases to determine, as an outcome, the subject's response to an agent, the outcome being communicable once known, preferably to a user having input the data.

Also provided herein is a computer program for use in a computer system as described, and the use of the results of such systems and programs in the prediction or determination of a subject's response to an agent, or in determining the suitability of a subject for a treatment regime or an intervention as described herein.

The predictive methods allow a number of therapeutic interventions and/or treatment regimens to be assessed for suitability and implemented for a given subject. The simplest of these can be the provision to the subject of motivation to implement a lifestyle change, for example, where the subject is a current smoker or sedentary, the methods can provide motivation to quit smoking and/or start exercising.

The manner of therapeutic intervention or treatment will be predicated by the nature of the polymorphism/s and the biological effect of the polymorphism/s. For example, where a resistance polymorphism is associated with a change in the expression of a gene, intervention or treatment may be directed to the restoration of normal expression of the gene, by, for example, administration of an agent capable of modulating the expression of the gene. Where a polymorphism is associated with decreased expression of a gene, therapy can involve administration of an agent capable of increasing the expression of the gene, and conversely, where a polymorphism is associated with increased expression of a gene, therapy can involve administration of an agent capable of decreasing the expression of the gene.

For example, in situations where a polymorphism is associated with upregulated expression of a gene, therapy utilizing, for example, RNAi or antisense methodologies can be implemented to decrease the abundance of mRNA and so decrease the expression of the gene. Alternatively, therapy can involve methods directed to, for example, modulating the activity of the product of the gene, thereby compensating for the abnormal expression of the gene.

Where a resistance polymorphism is associated with decreased gene product function or decreased levels of expression of a gene product, therapeutic intervention or treatment can involve augmenting or replacing of the function, or supplementing the amount of gene product within the subject for example, by administration of the gene product or a functional analogue thereof. For example, where a polymorphism is associated with decreased enzyme function, therapy can involve administration of active enzyme or an enzyme analogue to the subject. Similarly, where a polymorphism is associated with increased gene product function, therapeutic intervention or treatment can involve reduction of the function, for example, by administration of an inhibitor of the gene product or an agent capable of decreasing the level of the gene product in the subject. For example, where a SNP allele or genotype is associated with increased enzyme function, therapy can involve administration of an enzyme inhibitor to the subject.

Likewise, when a responsive polymorphism is associated with upregulation of a particular gene or expression of an enzyme or other protein, therapies can be directed to mimic such upregulation or expression in a subject lacking the resistive genotype, and/or delivery of such enzyme or other protein to such subject.

Further, when a responsive polymorphism is associated with downregulation of a particular gene, or with diminished or eliminated expression of an enzyme or other protein, desirable therapies can be directed to mimicking such conditions in a subject that lacks the responsive genotype.

The method can further include transmitting the report to the human or to a medical practitioner.

### Example 7

Design of Therapeutic Agents

The relationship between the various polymorphisms identified above and the responsiveness of a subject to an agent, or susceptibility (or otherwise) of a subject to a disease or condition associated with CAD also has application in the design and/or screening of candidate therapeutics. This is particularly the case where the association between a resistance polymorphism is manifested by either an upregulation or downregulation of expression of a gene. In such instances, the effect of a candidate therapeutic on such upregulation or downregulation is readily detectable.

Similarly, where the polymorphism is one which when present results in a physiologically active concentration of an expressed gene product outside of the normal range for a subject (adjusted for age and sex), and where there is an available prophylactic or therapeutic approach to restoring levels of that expressed gene product to within the normal range, subject subjects can be screened to determine the likelihood of their benefiting from that restorative approach. Such screening involves detecting the presence or absence of the polymorphism in the subject by any of the methods described herein, with those subjects in which the polymorphism is present being identified as subjects likely to benefit from treatment.

Kits

Also provided herein are kits useful for screening nucleic acid isolated from one or more subjects for allelic variation of any one of the mitochondrial transcription factor genes, and in particular for any of the SNPs described herein, wherein the kits may comprise at least one oligonucleotide selectively hybridizing to a nucleic acid comprising any one of the one or more of which are SNPs described herein and instructions for using the oligonucleotide to detect variation in the nucleotide corresponding to the SNP of the isolated nucleic acid.

Also provided is a diagnostic kit for detecting one or more polymorphisms in a genetic sample from a human subject refractory to statin treatment, further comprising a polymerase chain reaction (PCR) primer set for amplifying nucleic acid fragments corresponding to the at least one probe. In non-limiting examples, the probe has a label capable of being detected, the label is detected by electrical, fluorescent or radioactive means, the probe is selected from the group of sense, anti-sense, and naturally occurring mutants, of the at least one probe; and/or, the probe is affixed to a substrate.

The kit can comprise one or more of the following: at least one primer and/or probe for determining a single polymorphism in a chromosomal copy of the gene, wherein the polymorphism is associated with the CAD or predisposition therefor; at least one primer and/or probe for determining a single polymorphism in two chromosomal copies of the gene, wherein the polymorphism is associated with the CAD or predisposition therefor; a combination of primers and/or probes for determining a combination of polymorphisms in a chromosomal copy of the gene, wherein the combination of polymorphisms is associated with the CAD or predisposition therefor; a combination of primers and/or probes for determining a combination of polymorphisms in two chromosomal copies of the gene, wherein the combination of polymorphisms is associated with the CAD or predisposition therefor; and/or, an enzyme for primer elongation, nucleotides and/or labeling groups. In certain embodiments, diagnostic kit further comprises computer software to analyze information of a hybridization of the at least one probe in the diagnostic kit.

One embodiment provides an oligonucleotide that specifically hybridizes to the isolated nucleic acid molecule, and wherein the oligonucleotide hybridizes to a portion of the isolated nucleic acid molecule comprising any one of the polymorphic sites in the CETP gene.

Suitable kits include various reagents for use in suitable containers and packaging materials, including tubes, vials, and shrink-wrapped and blow-molded packages. Materials suitable for inclusion in an exemplary kit comprise one or more of the following: gene specific PCR primer pairs (oligonucleotides) that anneal to DNA or cDNA sequence domains that flank the genetic polymorphism/s of interest, reagents capable of amplifying a specific sequence domain in either genomic DNA or cDNA without the requirement of performing PCR; reagents required to discriminate between the various possible alleles in the sequence domains amplified by PCR or non-PCR amplification (e.g., restriction endonucleases, oligonucleotide that anneal preferentially to one allele of the polymorphism, including those modified to contain enzymes or fluorescent chemical groups that amplify the signal from the oligonucleotide and make discrimination of alleles more robust); reagents required to physically separate products derived from the various alleles (e.g. agarose or polyacrylamide and a buffer to be used in electrophoresis, HPLC columns, SSCP gels, formamide gels or a matrix support for MALDI-TOF).

The specific methods described herein are representative of various embodiments or preferred embodiments and are exemplary only and not intended as limitations on the scope of the invention. Other objects, aspects, examples and embodiments will occur to those skilled in the art upon consideration of this specification, and are encompassed within the spirit of the invention as defined by the scope of the claims. It will be readily apparent to one skilled in the art that varying substitutions and modifications can be made to the invention disclosed herein without departing from the scope and spirit of the invention.

The invention illustratively described herein suitably can be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of may be replaced with either of the other two terms in the specification, thus indicating additional examples, having different scope, of various alternative embodiments of the invention. Also, the terms “comprising”, “including”, containing“, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed.

Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention.

1. A method for determining whether a human subject has an altered risk for developing a coronary artery disease (CAD), comprising:
testing nucleic acid from the human subject to determine the presence of at least one single nucleotide polymorphism (SNP) in the cholesteryl ester transfer protein (CETP) gene,
the at least one SNP comprising one or more of:
a) rs247616 [SEQ ID N0:31], rs5883 [SEQ ID N0:34], rs3764261 [SEQ ID N0:33], and rs9930761 [SEQ ID N0:35],
b) one or more SNPs in linkage disequilibrium with the SNP/s of (a), and
c) combinations of (a) and (b);
wherein the presence of a minor allele of the SNP being detected indicates that the human subject has an altered risk for developing CAD.

2. The method of claim 1, wherein, wherein the SNP is rs247616 [SEQ ID N0:31].

3. The method of claim 1, wherein, wherein the SNP is rs5883 [SEQ ID N0:34].

4. The method of claim 1, wherein, wherein the SNP is rs5883 [SEQ ID N0:33].

5. The method of claim 1, wherein, wherein the SNP is rs5883 [SEQ ID N0:35].

6. The method of claim 1, wherein, wherein the SNP is rs5883 [SEQ ID N0:31].

7. The method of claim 1, wherein, wherein the SNP are rs247616 [SEQ ID N0:31] and rs3764261 [SEQ ID N0:33].

8. The method of claim 1, wherein, wherein the SNP are rs247616 [SEQ ID N0:31] and rs9930761 [SEQ ID N0:35].

9. The method of claim 1, wherein, wherein the SNPs are rs5883 [SEQ ID N0:34] and rs9930761 [SEQ ID N0:35].

10. The method of claim 1, wherein the testing step further comprises detecting whether the human subject is homozygous for the SNP.

11-16. (canceled)

17. The method of claim 1, wherein CAD comprises at least: an altered response to a cholesterol ester transfer protein (CETP) inhibitor.

18. The method of claim 1, wherein CAD comprises one or more of: arterial disease, atheroma, atherosclerosis, arteriosclerosis, coronary artery disease, arrhythmia, angina pectoris, congestive heart disease, myocardial infarction, stroke, transient ischemic attack (TIA), aortic aneurysm, cardiopericarditis, infection and/or inflammation of heart tissue, vascular and clotting problems, insufficiencies, and combinations thereof.

19. The method of claim 1, wherein at least one additional SNP of the CETP gene that is in high linkage disequilibrium with rs247616 [SEQ ID N0:31] is tested,
wherein the at least one additional SNP is selected from several SNPs including: rsl 73539 [SEQ ID N0:32], rs3726432 [SEQ ID N0:53], and rs3764261[SEQ ID N0:33].

20. The method of claim 1,wherein at least one additional SNP of the CETP gene that is in high linkage disequilibrium with rs9930761 [SEQ ID N0:35] and/or rs5883 [SEQ ID N0:34] is tested, wherein the at least one additional SNP is selected from: rs12720873 [SEQ ID N0:36] and rs11644475 [ SEQ ID N0:37].

21. The method of claim 1, wherein the human subject is a male.

22. The method of claim 1, wherein the human subject is a healthy human subject lacking other CAD risk factors.

23. The method of claim 1, wherein the human subject is a human subject already progressing toward CAD with at least one other CAD risk factor.

24. The method of claim 1, wherein the human subject is evaluated for the human's medical history, family history, age, gender, socio-economic status, race, ethnicity, smoking, plasma cholesterol levels, plasma triglyceride levels, plasma lipids, plasma glucose, plasma insulin, plasma lipoproteins, or coronary artery calcification using electron-beam computer tomography.

25. The method of claim 1,wherein the method comprises performing a procedure selected from:
chain terminating sequencing, restriction digestion, allele-specific polymerase reaction, single-stranded conformational polymorphism analysis, genetic bit analysis, temperature gradient gel electrophoresis, ligase chain reaction, ligase/polymerase genetic bit analysis, allele specific hybridization, size analysis, nucleotide sequencing, 5′ nuclease digestion, primer specific extension, oligonucleotide microarray analysis, oligonucleotide ligation assay, or mass spectrophotometry.

26. The method of claim 1, wherein the method comprises using a probe or primer that hybridizes under high stringency conditions to a nucleic acid sequence spanning the nucleotide.

27-64. (canceled)


SEQUENCE LISTING (53 sequences)

SEQ ID NO: 1 - DNA, 23 bp, Artificial Sequence
  Description of Artificial Sequence Synthetic primer
cctgtggtcccagttacttagga

SEQ ID NO: 2 - DNA, 22 bp, Artificial Sequence
  Description of Artificial Sequence Synthetic primer
ccccaatctgtagtctttgcca

SEQ ID NO: 3 - DNA, 20 bp, Artificial Sequence
  Description of Artificial Sequence Synthetic primer
gactcaacaacagggccaca

SEQ ID NO: 4 - DNA, 31 bp, Artificial Sequence
  Description of Artificial Sequence Synthetic primer
acttcgattaaaagagttctggagatgggtt

SEQ ID NO: 5 - DNA, 34 bp, Artificial Sequence
  Description of Artificial Sequence Synthetic primer
cgtcccgcgccgcccctgtcggtaggcatcttgg

SEQ ID NO: 6 - DNA, 21 bp, Artificial Sequence
  Description of Artificial Sequence Synthetic primer
acctgtcggtaggcatcaggt

SEQ ID NO: 7 - DNA, 19 bp, Artificial Sequence
  Description of Artificial Sequence Synthetic primer
cagggcaatcaaggcatcc

SEQ ID NO: 8 - RNA, 180 nt, Homo sapiens
ggucauuucaucuacaagaaugucucagaggaccucccccuccccaccuucucgcccacacugcugggggacucccgcaugcuguacuucugguucucugagcgagucuuccacucgcuggccaagguagcuuuccaggauggccgccucaugcucagccugaugggagacgaguucaag

SEQ ID NO: 9 - RNA, 180 nt, Homo sapiens
ggucauuucaucuacaagaaugucucagaggaccucccccuccccaccuucucgcccacacugcugggggacucccgcaugcuguacuucugguucucugagcgagucuuucacucgcuggccaagguagcuuuccaggauggccgccucaugcucagccugaugggagacgaguucaag

SEQ ID NO: 10 - DNA, 20 bp, Artificial Sequence
  Description of Artificial Sequence Synthetic primer
ttgaatgagtgaaagccccg

SEQ ID NO: 11 - DNA, 17 bp, Artificial Sequence
  Description of Artificial Sequence Synthetic primer
gacgctggctggaccct

SEQ ID NO: 12 - DNA, 506 bp, Homo sapiens
gtcaatggactgccctctgcaggggcagggctggtggtcagctggggcggggtgggagctggaggtccgtggtcaccagctgccctgactaatgtcgttacttgaatataaccctgtgaaggcaggaaccacgtctgtctggttcacttcccacggtggttgagacatagtgggcactccggaagtatttgttgaatgagtgaaagccccgctgggggaaactgggtacagctctttcctcagtttccccatctgcactctgggctgaatgctggggctcctcccaatctccctgaagctggaccygagcccagtagggacacacagggtccagccagcgtcctggcttcctccagggtcatttcatctacaagaatgtctcagaggacctccccctccccaccttctcgcccacactgctgggggactcccgcatgctgtacttctggttctctgagcgagtcttycactcgctggccaaggtagctttccaggatggccgcctc

SEQ ID NO: 13 - DNA, 13 bp, Artificial Sequence
  Description of Artificial Sequence Synthetic oligonucleotide
ggacccgagccca

SEQ ID NO: 14 - DNA, 13 bp, Artificial Sequence
  Description of Artificial Sequence Synthetic oligonucleotide
ggacctgagccca

SEQ ID NO: 15 - DNA, 51 bp, Homo sapiens
acttctggttctctgagcgagtcttycactcgctggccaaggtagctttcc

SEQ ID NO: 16 - DNA, 19 bp, Artificial Sequence
  Description of Artificial Sequence Synthetic primer
caccttctcgcccacactg

SEQ ID NO: 17 - DNA, 20 bp, Artificial Sequence
  Description of Artificial Sequence Synthetic primer
gaactcgtctcccatcaggc

SEQ ID NO: 18 - DNA, 20 bp, Artificial Sequence
  Description of Artificial Sequence Synthetic primer
gcctgatgggagacgagttc

SEQ ID NO: 19 - DNA, 30 bp, Artificial Sequence
  Description of Artificial Sequence Synthetic primer
gctgtacttctggttctctgagcgagtctt

SEQ ID NO: 20 - DNA, 24 bp, Artificial Sequence
  Description of Artificial Sequence Synthetic primer
gaaagctaccttggccagcgagtg

SEQ ID NO: 21 - DNA, 20 bp, Artificial Sequence
  Description of Artificial Sequence Synthetic primer
ggagtcccatcacaaggcag

SEQ ID NO: 22 - DNA, 21 bp, Artificial Sequence
  Description of Artificial Sequence Synthetic primer
cctgatgggagacgagttcaa

SEQ ID NO: 23 - DNA, 21 bp, Artificial Sequence
  Description of Artificial Sequence Synthetic primer
tacgagacatgacctcaggga

SEQ ID NO: 24 - DNA, 20 bp, Artificial Sequence
  Description of Artificial Sequence Synthetic primer
cccgtcatcacagcctccta

SEQ ID NO: 25 - DNA, 21 bp, Artificial Sequence
  Description of Artificial Sequence Synthetic primer
cattcagcccagagtgcagat

SEQ ID NO: 26 - DNA, 24 bp, Artificial Sequence
  Description of Artificial Sequence Synthetic primer
gccctgactaatgtcgttacttga

SEQ ID NO: 27 - DNA, 17 bp, Artificial Sequence
  Description of Artificial Sequence Synthetic primer
cccatgttgcccaggct

SEQ ID NO: 28 - DNA, 21 bp, Artificial Sequence
  Description of Artificial Sequence Synthetic primer
tgcatgcaccggtaatcagaa

SEQ ID NO: 29 - DNA, 19 bp, Artificial Sequence
  Description of Artificial Sequence Synthetic primer
cccaggtctccagcactgc

SEQ ID NO: 30 - DNA, 21 bp, Artificial Sequence
  Description of Artificial Sequence Synthetic primer
cggcggtgcatacctatagtc

SEQ ID NO: 31 - DNA, 52 bp, Homo sapiens
  modified_base (27)..(27): a, c, t or g
acataaaattgatcattttagccattngaagtgtacagttttgtggcgttta

SEQ ID NO: 32 - DNA, 52 bp, Homo sapiens
  modified_base (27)..(27): a, c, t or g
gggcagcctcttgagcctaagtaggcncagagactgtccagcatactattat

SEQ ID NO: 33 - DNA, 52 bp, Homo sapiens
  modified_base (27)..(27): a, c, t or g
cccttcacctgtcggtaggcatctggnttgtctgctatctcattcactcttc

SEQ ID NO: 34 - DNA, 52 bp, Homo sapiens
  modified_base (27)..(27): a, c, t or g
tacttctggttctctgagcgagtcttncactcgctggccaaggtagctttcc

SEQ ID NO: 35 - DNA, 52 bp, Homo sapiens
cctcccaatctccctgaagctggaccygagcccagtagggacacacagggtc

SEQ ID NO: 36 - DNA, 52 bp, Homo sapiens
gtccttttttgtgctctgacaaccccrtcccccagcttcaaccttatggcag

SEQ ID NO: 37 - DNA, 52 bp, Homo sapiens
gacgaaccgcagacaaaactcctcagrcactgagttaaagaaggaaagagtt

SEQ ID NO: 38 - DNA, 52 bp, Homo sapiens
acctggctcagatctgaaccctaactygaaccccagtgattctgggtctcag

SEQ ID NO: 39 - DNA, 52 bp, Homo sapiens
ccaatgatctcagaggctgtatacccmcccagagttattttatgcatatcaa

SEQ ID NO: 40 - DNA, 52 bp, Homo sapiens
tggaaacagctcatttagtggttgagyctccaattatctgctctctcttaga

SEQ ID NO: 41 - DNA, 52 bp, Homo sapiens
ttgattggcagagcagctccgagtccrtccagagcttcctgcagtcaatgat

SEQ ID NO: 42 - DNA, 52 bp, Homo sapiens
  modified_base (27)..(27): a, c, t or g
acacacataccacatgccatctggatnccctagcagcccagccccacccact

SEQ ID NO: 43 - DNA, 52 bp, Homo sapiens
gctggaaccctggtgtctcctccagcrtggtggaagttgggttaggagtacg

SEQ ID NO: 44 - DNA, 52 bp, Homo sapiens
tacttctggttctctgagcgagtctttcactcgctggccaaggtagctttcc

SEQ ID NO: 45 - DNA, 52 bp, Homo sapiens
cctcccaatctccctgaagctggacccgagcccagtagggacacacagggtc

SEQ ID NO: 46 - DNA, 52 bp, Homo sapiens
  modified_base (27)..(27): a, c, t or g
gtagtatgtatgtctccagagagacanatgttcacagaacacccagtaaggg

SEQ ID NO: 47 - DNA, 52 bp, Homo sapiens
gcctcctggtcgcactgttgctgctgytgctactgagccgccgccgcacgcg

SEQ ID NO: 48 - DNA, 52 bp, Homo sapiens
cccaatggagggtcaaattatcatcgyttttttatttcaggattacaccaaa

SEQ ID NO: 49 - DNA, 52 bp, Homo sapiens
cagtcctctcggtcctcagtccagtgkgggtttccctgggctgaagtcaagg

SEQ ID NO: 50 - DNA, 52 bp, Homo sapiens
  modified_base (27)..(27): a, c, t or g
gctcaaatactggctttctgtccctangtgacctggagcttgtagtttaact

SEQ ID NO: 51 - DNA, 52 bp, Homo sapiens
gagaattaacttcacagtggagaaatstgacaaatacgacctccgccaagtg

SEQ ID NO: 52 - DNA, 52 bp, Homo sapiens
  modified_base (27)..(27): a, c, t or g
gtgccctgggagggagatgggctgagnggagctgtcatcaccccctcctgac

SEQ ID NO: 53 - DNA, 52 bp, Mus musculus
ggagcctgtgcatgtggcatgtcacaycatctctctagtcactttccacagt